(19) United States
(12) Patent Application Publication
(10) Pub. No.: US 2010/0008956 A1
(43) Pub. Date: Jan. 14, 2010

(54) COMPOSITION AND COMBINATIONS OF CARBOXYLIC ACID LOSARTAN IN DOSAGE FORMS

(76) Inventor: Jie Du, Lansdale, PA (US)

Correspondence Address: FOX ROTHSCHILD LLP;PRINCETON PIKE CORPORATE CENTER, 2000 Market Street, Tenth Floor, Philadelphia, PA, 19103, US

(21) Appl. No.: 12/169,430

(22) Filed: Jul. 8, 2008

(51) Int. Cl. A61K 9/00; C07D 403/08; A61P 9/12; A61K 31/4155

(52) U.S. Cl. 424/400; 548/252; 514/381

(57) ABSTRACT

Compositions comprising a Carboxylic Acid Losartan in a dosage form are provided. Such compositions may be employed for treatment of hypertension, congestive heart failure, diabetic nephropathy, and myocardial infarction. The compositions may further include one or more additional therapeutic agents based on the condition to be treated.

### FIELD OF INVENTION

This invention relates to compositions and combination of Carboxylic Acid Losartan (CAL) in dosage forms. The invention further relates to methods of using such compositions and combination.

### BACKGROUND

Angiotensin II [formed from Angiotensin I in a reaction catalyzed by angiotensin converting enzyme (ACE, kininase II)], is a potent vasoconstrictor, the primary vasoactive hormone of the renin-angiotensin system and an important component in the pathophysiology of hypertension. It also stimulates aldosterone secretion by the adrenal cortex. There is also an AT₂ receptor found in many tissues but it is not known to be associated with cardiovascular homeostasis.

Losartan is an orally active agent with the following chemical formula:

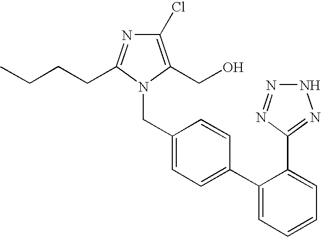

Losartan undergoes substantial first-pass metabolism by cytochrome P450 enzymes and is converted to one active metabolite in addition to several inactive metabolites. Losartan and its principal active metabolite, the 5-carboxylic acid designated as EXP3174 and referred to here as Carboxylic Acid Losartan (CAL), block the vasoconstrictor and aldosterone-secreting effects of angiotensin II by selectively blocking the binding of angiotensin II to the AT₁ receptor found in many tissues, (e.g., vascular smooth muscle, adrenal gland). CAL is responsible for most of the angiotensin II receptor antagonism that follows losartan treatment.

Currently, only losartan is commercially available for treatment of hypertension. However, because liver isoenzyme P450 systems play a major role in the metabolism of losartan, losartan has many cited drug-drug interactions due to the competitive effect of liver metabolism. In addition, studies have shown that CAL is 10 to 40 times more potent by weight than losartan. Thus, CAL may provide hypertensive patient with an improved treatment at lower dosages and lower liver toxicity.

Accordingly, there is a need in the art to develop a dosage form containing Carboxylic Acid Losartan (CAL).

### SUMMARY OF INVENTION

In one aspect, a dosage form is provided, where the dosage form comprises a composition comprising a therapeutically effective amount of carboxylic acid losartan, or its pharmaceutically acceptable salts, isomers, polymorphs, hydrates, solvates, or metabolites (hereinafter referred to collectively as “CAL”). Such dosage form may comprise between about 1 and about 120 mg of CAL. In some embodiments, the composition may also include a therapeutically effective amount of one or more additional active agents.

The dosage form may be formulated as either an immediate release dosage form or a modified release dosage form. Administration of the immediate release dosage form may result in the AUC between about 185 and about 7920 ng.h/mL, Tmax between about 0.5 and 6 hours, and Cmax between about 25 and about 1000 ng/mL. Administration of the modified release dosage form may result in the AUC between about 185 and about 7920 ng.h/mL, Tmax is between about 3 and about 14 hours, and Cmax is between about 25 and about 800 ng/mL.

In another aspect, methods of treating hypertension, congestive heart failure, diabetic nephropathy, or myocardial infarction is provided. Such methods comprise administering a therapeutically effective amount of a composition comprising CAL. In addition to CAL, the composition may further comprise one or more additional therapeutic agents based on the condition being treated. In one non-limiting embodiment, the composition of CAL and at least one cholesterol lowering drug is administered in a dosage form to treat hypertension patients with high cholesterol. In another non-limiting embodiment, the composition of CAL and at least one lipid lowering drug is administered in a dosage form to treat hypertension patients with lipid abnormalities. In another non-limiting embodiment, the composition of CAL and a lipid lowering agent and a cholesterol lowering agent is administered in a dosage form to treat hypertension patients with lipid and cholesterol abnormalities.

In some embodiments, the amount of CAL is administered to a patient is personalized based on individual patient's weight. By way of non-limiting example, between about 1 and about 70 mg of CAL per dosage unit may be administered to patients weighing less than 170 lbs, between about 3 and about 100 mg of CAL per dosage unit may be administered to patients weighing between about 150 and about 300 lbs, and between about 7 and about 120 mg of CAL per dosage unit is administered to patients weighing more than 270 lbs.

In yet another aspect, a dosage form comprising a composition comprising a therapeutically effective amount of CAL and a therapeutically effective amount of cholesterol-lowering drug, lipid-lowering drug, or both is provided. In some embodiments, such dosage form may comprise between about 1 and about 100 mg of carboxylic acid losartan and a lipid-lowering drug. In other embodiments, such dosage form may comprise between about 1 and about 100 mg of carboxylic acid losartan and a cholesterol-lowering drug. In other embodiments, such dosage form may comprise between about 1 and about 90 mg of carboxylic acid losartan and a cholesterol-lowering drug and a lipid-lowering agent.

### BRIEF DESCRIPTION OF THE FIGURES

FIG. 1 presents an exemplary pharmacokinetic profile for an immediate release oral solid dosage comprising CAL.

FIG. 2 presents an exemplary pharmacokinetic profile after intravenous administration of a parenteral dosage form comprising CAL.

FIG. 3 presents an exemplary pharmacokinetic profile for an extended release dosage form comprising CAL.

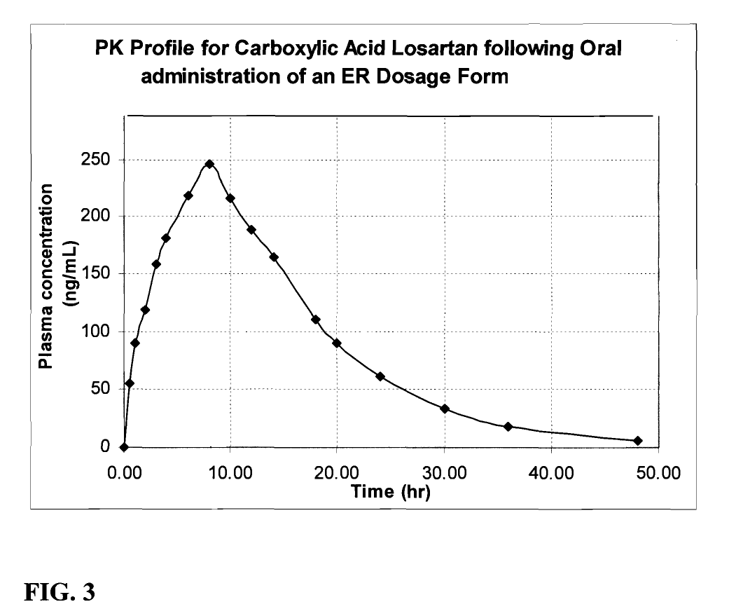

### DETAILED DESCRIPTION

In one general aspect, compositions comprising Carboxylic Acid Losartan (CAL) or a pharmaceutically acceptable salt, isomer, polymorph, hydrate, solvate, or metabolite thereof as an active agent in a dosage form are generally provided. In another aspect, methods of using such dosage forms to treat diseases caused by Angiotensin II activity are provided.

Definitions:

The term “active agent” means a compound, element, or mixture that when administered to a patient, alone or in combination with another compound, element, or mixture, confers, directly or indirectly, a physiological effect on the patient. The indirect physiological effect may occur via a metabolite or other indirect mechanism. When the active agent is a compound, then salts, solvates (including hydrates) of the free compound or salt, crystalline forms, non-crystalline forms, and any polymorphs of the compound are contemplated herein. Compounds may contain one or more asymmetric elements such as stereogenic centers, stereogenic axes and the like, e.g., asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, all optical isomers in pure form and mixtures thereof are encompassed. In addition, compounds with carbon-carbon double bonds may occur in Z- and E-forms, with all isomeric forms of the compounds. In these situations, the single enantiomers, i.e., optically active forms can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column. All forms are contemplated herein regardless of the methods used to obtain them.

The term “pharmaceutically acceptable carrier” refer to a non-toxic, inert solid, semi-solid or liquid filler, diluent, encapsulating material or formulation auxiliary of any type. Many different pharmaceutically acceptable carriers are known and disclosed, for example, in Remington's Pharmaceutical Sciences, Lippincott Williams & Wilkins; 21 edition (May 1, 2005). Some examples of the materials that can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol and phosphate buffer solutions, as well as other non-toxic compatible substances used in pharmaceutical formulations. Other non-limiting examples are also presented throughout the instant disclosure.

The term “pharmaceutically acceptable salts” include derivatives of the active agent (e.g. CAL), wherein the parent compound is modified by making non-toxic salts thereof, and further refers to pharmaceutically acceptable solvates, including hydrates, of such compounds and such salts. Also included are all crystalline, amorphous, and polymorph forms. The list of suitable salts may be found in Remington's Pharmaceutical Sciences, Lippincott Williams & Wilkins, 21^{st} edition, (May 1, 2005). Carboxylic acid losartan salts include base addition salts. Suitable base addition salts include salts with inorganic bases, for example metal hydroxides or carbonates of alkali metals, alkaline earth metals or transition metals, or with organic bases, for example ammonia, basic amino acids such as arginine and lysine, amines, e.g., methylamine, dimethylamine, trimethylamine, triethylamine, ethylamine, diethylamine, ethylenediamine, ethanolamine, diethanolamine, 1-amino-2-propanol, 3-amino-1-propanol or hexamethylenetetraamine, saturated cyclic amines having 4 to 6 ring carbon atoms, such as piperidine, piperazine, pyrrolidine and morpholine, and other organic bases, for example N-methylglucamine, kreatine and tromethamine, and quaternary ammonium compounds such as tetramethylammonium and the like. Suitable salts with organic bases are formed with amino acids. Suitable salts with inorganic bases are formed with sodium, potassium, magnesium, and calcium cations. Additional salts of carboxylic acid losartan also include acid addition salts, such as, the moroxydine salt, cinnarizine-salt, and sodium salt.

The term “salts” as used herein, denotes acidic salts formed with inorganic (metallic) and organic acids, as well as basic salts formed with inorganic (metallic) and organic bases.

The term “dosage form” means a unit of administration of instant compositions. Examples of dosage forms include, but are not limited to, tablets, capsules, powders, injections, suspensions, non-sterile liquids, sterile liquids, emulsions, creams, ointments, suppositories, inhalable forms, transdermal forms, and the like.

The term “rapid release system” or “immediate release system” means a system where the active agent is released immediately into the blood, i.e., immediately available for absorption. An immediate release dosage form is one in which the release properties of the active agent from the dosage form are essentially unmodified. An immediate release dosage form results in delivery of greater then or equal to about 75% the carboxylic acid losartan within about 3 hours of administration, specifically within about 1 hour of administration. Although an immediate-release dosage form may contain optional excipients so long as the excipients do not significantly extend the release time of the carboxylic acid losartan.

The term “modified release system” means a system where the active agent is released over time at the same or different rate instead of being immediately released. The modified release systems encompass controlled release systems (systems in which the release rate may be controlled or modified over time), sustained and extended release systems (systems in which the active agent is released at the constant or variable rate to maintain its blood level over required periods of time such as hours, days, or months), delayed release systems (systems in which there is a time delay between administration of the composition and the release of the active agent), or repeat action systems (systems in which one dose of drug is released either immediately or some time after administration and further doses are released at a later time.)

The term “therapeutically effective amount” means a quantity of the active agent which, when administered to a patient, is sufficient to result in an improvement in patient's condition. The improvement does not mean a cure and may include only a marginal change in patient's condition. It also includes an amount of the active agent that prevents the condition or stops or delays its progression.

The term “treating” or “treatment” refers to executing a protocol, which may include administering one or more drugs to a patient (human or otherwise), in an effort to alleviate signs or symptoms of the disease. Alleviation can occur prior to signs or symptoms of disease appearing, as well as after their appearance. Thus, “treating” or “treatment” includes “preventing” or “prevention” of the disease. In addition, “treating” or “treatment” does not require complete alleviation of signs or symptoms, does not require a cure, and specifically includes protocols which have only a marginal effect on the patient.

The term “bioavailability” means the extent or rate at which an active agent is absorbed into a living system or is made available at the site of physiological activity. For active agents that are intended to be absorbed into the bloodstream, bioavailability data for a given formulation may provide an estimate of the relative fraction of the administered dose that is absorbed into the systemic circulation. “Bioavailability” can be characterized by one or more pharmacokinetic parameters.

The term “efficacy” means the ability of an active agent administered to a patient to produce a therapeutic effect in the patient.

The term “safety” means the incidence or severity of adverse events associated with administration of an active agent, including adverse effects associated with patient-related factors (e.g., age, gender, ethnicity, race, target illness, abnormalities of renal or hepatic function, co-morbid illnesses, genetic characteristics such as metabolic status, or environment) and active agent-related factors (e.g., dose, plasma level, duration of exposure, or concomitant medication).

Pharmacokinetic profile describes the in vivo characteristics of the active agent over time. For purposes of this disclosure, Cmax refers to maximum concentration of the active agent in plasma and Cn refers to concentration of the active agent in plasma after a certain number of hours, i.e. n hours, after administration of the active agent. Furthermore, Tₘₐₓ refers to the time at which the measured concentration of the active agent in plasma is the highest after administration of the active agent. Finally, AUC is the area under the curve of a graph of the concentration of the active agent in plasma over time measured between two points in time. For example AUC₀₋₂₄ or AUC₂₄ means area under the curve of active agent plasma concentration over time calculated between about 0 hours and about 24 hours. AUC₀₋₇₂ or AUC₇₂ means area under the curve of active agent plasma concentration over time calculated between about 0 hours and about 72 hours.

In one embodiment, pharmacokinetic profile of a carboxylic acid losartan composition is determined by an in vivo bioavailability study to determine a pharmacokinetic parameter for the carboxylic acid losartan composition. For example, the pharmacokinetic parameters for a carboxylic acid losartan composition of the present invention and for a comparator drug can be measured in a single dose bioavailability study using a two-period, two-sequence crossover design. Alternately, a four-period, replicate design crossover study may also be used. Single doses of the test composition and comparator drug are administered and blood or plasma levels of the active agent are measured over time. Pharmacokinetic parameters characterizing the rate and extent of active agent absorption are evaluated statistically.

The area under the plasma concentration-time curve from time zero to the time of measurement of the last quantifiable concentration (AUC₀₋ₜ) and to infinity (AUC_{0-∞}), Cₘₐₓ, and Tₘₐₓ can be determined according to standard techniques. For statistical analysis of pharmacokinetic data, the logarithmic transformed AUC₀₋ₜ, AUC_{0-∞}, or Cₘₐₓ data can be analyzed statistically using analysis of variance.

Compositions:

Active agent:

The active agent in the instant compositions is Carboxylic acid losartan which is an active metabolite of Losartan. It has the chemical name 2-n-butyl-4-chloro-1-[2′-(H-tetrazol-5-yl)-1,1′-biphenyl-4-yl)methyl]-1H-imidazole-5-carboxylic acid and has the following formula:

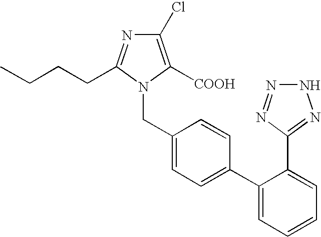

For the purposes of the instant disclosure, the terms “carboxylic acid losartan” or “CAL” include carboxylic acid losartan of the above formula, as well as, various variations as described in detail under the definition of the term “active agent.” Specifically, these terms include carboxylic acid losartan's pharmaceutically acceptable salts, isomers, polymorphs, hydrates, solvates, metabolites, and combinations thereof.

Methods of Treatment:

In another aspect, methods of treatment of hypertension, congestive heart failure, diabetic nephropathy, or myocardial infarction are provided. Such methods comprise administering a therapeutically effective amount of a composition in a dosage form comprising carboxylic acid losartan.

The amount of CAL in the composition may vary depending on the subject being treated, the severity of the disease state and the manner of administration, and may be determined routinely by one of ordinary skill in the art. The dose, dose frequency, and dosage form may also vary according to the age, body weight, and response of the individual patient. In one specific embodiment, a composition in a solid dosage form comprising between about 1 mg and 120 mg of CAL may be administered once daily to a patient suffering from hypertension. Although the effect of CAL may not be noticeable until week 6, in particular embodiments, an increase in dosage or increase in frequency may be required if the effect of CAL is not present within one week of the initial administration.

The instant methods also contemplate that a therapeutically effective amount of one or more additional active agents may be administered in combination with CAL. These additional agents may be administered in any dosage form suitable for the formulation as are well known in the art. The one or more agents may be incorporated in the same dosage form as CAL or may be administered in a separate dosage form. Preferably, all therapeutic agents are presented in a combined form to facilitate patient compliance.

A person with ordinary skill in the art would undoubtedly be able to select an appropriate additional active agents based upon the requirements of the patient and the severity and type of the condition being treated. For example, for treatment of patients suffering from hypertension, the composition may include diuretics, beta-blockers, angiotensin converting (“ACE”) inhibitors, calcium channel blockers, alpha-blockers, alpha-beta blockers, vasodilators, alpha antagonists, adrenergic neuron blockers, or a combination thereof. For treatment of patients suffering from congestive heart failure, the compositions may include one or more of diuretics, ACE inhibitors, digoxin, vasodilators, beta blockers, statins, or a combination thereof. For treatment of patients with diabetic nephropathy, such additional therapeutic agents can include one or more diuretics. For treatment of patients suffering from myocardial infarction, the composition may include ACE inhibitors, diuretics, vasodilators, beta blockers, anticoagulants, or a combination thereof.

The specific compounds within each class of drugs identified above are known in the art. Following specific examples of these drugs are intended to be purely illustrative and not limiting in any manner. Suitable examples of diuretics include, but are not limited to, chlorothiazide, bendroflumethiazide, chlorthalidone, hydrochlorothiazide, hydroflumethiazide, metolazone, methyclothiazide, bumetanide, ethacrynic, amiloride, and triamterene. Suitable examples of beta-blockers include, but are not limited to, propranolol, timolol, and metoprolol. Examples of suitable ACE inhibitors, include, but are not limited to, enalapril, lisinopril, quinapril, ramipril, and benazepril. Suitable calcium channel blockers include, but are not limited to, diltiazem, nimodipine, nifedipine, nicardipine, felodipine, isradipine, and amlodipine. Exemplary alpha-blockers may include, but are not limited to, prazosin, terazosin, doxazosin, phenoxybenzamine and phentolamine, whereas suitable alpha-beta blockers include labetol and celiprolol.

Suitable examples of vasolidators include, but are not limited to, hydralazine, minoxidil, diazoxide and nitroprussid. Examples of alpha antagonists include methyldopa, clonidine, and guanfacine. Exemplary adrenergic neuron blockers include, but are not limited to, guantacine, guanethidine, gunadrel, and reserpine. Examples of suitable statins are presented below. Suitable beta-blockers include, but are not limited to, nadolol, oxprenolol, penbutolol, acebutolol, atenolol, and betaxolol. Exemplary anticoagulants include, but are not limited to, warfarin, acenocoumarol, and heparin.

In embodiments where the hypertension is associated with, at least in part, high cholesterol and/or high plasma lipid content, the composition may include Carboxylic acid losartan in combination with cholesterol-lowering drugs, lipid-lowering drugs, or both. Suitable drugs include, but are not limited to, statins such as, atorvastatin, fluvastatin, lovastatin, pravastatin, rosuvastatin calcium, simvastatin; resins (also known as bile acid sequestrant or bile acid-binding drugs), such as cholestyramine, colestipol, colesevelam hcl; fibrates (fibric acid derivatives), such as gemfibrozil, fenofibrate, clofibrate; niacin (nicotinic acid), such as polygel extended release niacin and prescription extended release niacin; and combination thereof. Other cholesterol-lowering drugs and lipid-lowering drugs are known and are disclosed for example, in International Patent Application No.: WO/2002/072104 and U.S. Patent Applciation No.: US2005/0101561A1.

### Specific Embodiments

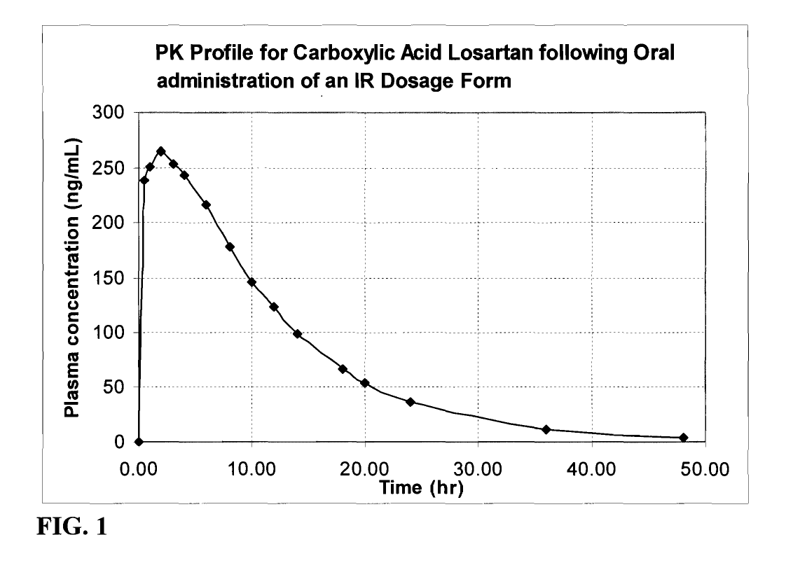

In one specific embodiment, an oral solid dosage form, preferable tablets or capsules, comprising CAL is provided. Such dosage may comprise between about 1 and about 120 mg of CAL, about 1.75 to about 112 mg of Cal, about 3.5 to about 56 mg, or about 7 to about 28 mg of CAL. Such dosage form may be an immediate-release dosage form with the following pharmacokinetic parameters: Tmax is about 0.5 to about 6 hr; Cₘₐₓ is about 25 to about 1000 ng/mL; and AUC is about 185 to about 7920 ng.h/mL. A typical pharmacokinetic plot for this embodiment is presented in FIG. 1.

In another embodiment, a liquid dosage form, preferable oral liquid, such as oral solution, oral suspension or emulsion, are provided. Such liquid dosages may comprise between about 1 and 100 mg of CAL, between about 1.75 and about 112 mg of CAL, between about 3.5 and about 56 mg, or between about 7 and about 28 mg of CAL.

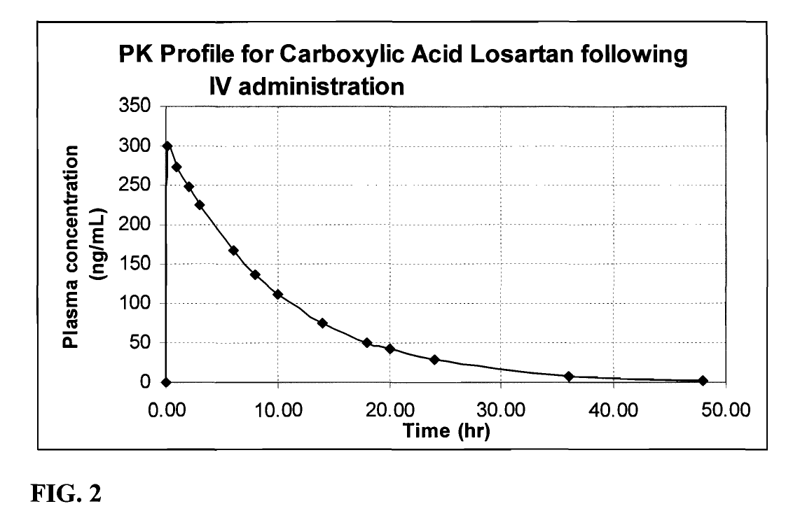

In yet another specific embodiment, an injectable dosage form, preferably for intravenous injection, comprising between about 1 and about 40 mg of CAL also provided. In addition to CAL, the intravenous injection form may comprises the following excipients: sodium chloride, dextrose, EDTA, buffering agent, ethanol, etc. This injectable dosage form may present the following pharmacokinetic profile: Tmax is about 2 to about 8 min., preferably 5 min; Cmax is about 30 to about 1200 ng/mL; and AUC₂₄ is about 240 to about 9600 ng.h/mL. A typical pharmacokinetic plot following intravenous administration of such dosage form is presented in FIG. 2.

A modified release dosage form is also provided in some embodiments. This dosage form presents the following pharmacokinetic profile: Tmax is between about 3 to about 14 hr; Cₘₐₓ is between about 25 and 800 ng/mL; and AUC is between about 185 and about 7920 ng.h/mL. A typical pharmacokinetic plot following intravenous administration of such dosage form is presented in FIG. 2.

In yet another specific embodiment, the instant composition comprises between about 1 and about 100 mg of CAL and at least one cholesterol lowering drug or its active metabolite. Preferably, the composition is administered in an oral dosage form with the following pharmacokinetic profile: Tmax is between about 0.5 and about 6 hr; Cₘₐₓ is between about 25 and about 830 ng/mL; and AUC is between about 185 and about 6600 ng.h/mL.

In yet another specific embodiment, the instant composition comprises between about 1 and about 100 mg of CAL and at least one lipid-lowering agent or its active metabolite. Preferably, the composition is administered in an oral dosage form with the following pharmacokinetic profile: Tmax is between about 0.5 and about 6 hr; Cmax is between about 25 and about 830 ng/mL; and AUC is between about 185 and about 6600 ng.h/mL.

Another specific embodiment provides the instant composition comprising between about 1 and about 90 mg of CAL and one lipid-lowering agent and one cholesterol-lowering agent. Preferably, the composition is administered in an oral dosage form with the following pharmacokinetic profile: Tmax is between about 0.5 and about 6 hr; Cₘₐₓ is between about 25 and about 750 ng/mL; AUC is between about 185 and about 5940 ng.h/mL

Another embodiment provides that the dosage form comprising CAL is administered as a personalized medicine based on body weight of patients according to Table 1 below: Patients may be categorized in various sizes based on their gender and body weight. Specifically, they are categorized in the following 3 sizes:

TABLE 1
Amount of CAL vs. patients weight.

| | Amount of |
|---|---|
| Patient's weight (lb) | CAL (mg) per dosage unit |
| less than 170 | About 1 to |
| | about 70 |
| 150-300 | About 3 to |
| | about 100 |
| over 270 | About 7 to |
| | about 120 |

Of course, a person with ordinary skill in the art will undoubtedly appreciate that the specific amount of CAL described in the preceding paragraphs are provided only as a benchmark, and such person will be capable of customizing them for specific patients depending on the subject being treated, the severity of the disease state and the manner of administration, among other factors.

Dosage Form:

The compositions may be administered in a dosage form, including, but not limited to, solid forms, liquid forms or non-solid forms, prepared for oral, parenteral, enteral or topical administration. Suitable examples of a solid form include, but are not limited to, tablets, pills, lozenges, dragees, powders, granules, capsules, etc. Solid forms may or may not include a pharmaceutically acceptable carrier. Suitable examples of liquid forms include, but not limited to, solutions, dispersions, emulsions, gels, syrups, slurries, suspensions, and so forth. Liquid formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet. Suitable non-solid forms for topical applications may be formulated in a suitable ointment, cream or lotion containing the active agents suspended or dissolved in one or more pharmaceutically acceptable carriers. Other suitable non-solid dosage forms include, but are not limited to, transdermal patches, inhalers, effervescent, implants, suppositories, etc.

Instant compositions may be delivered by rapid release systems or modified release systems. Various approaches are known and used in the art to prepare immediate release systems or modified release systems. Many of these methods are disclosed, for example, in Remington's Pharmaceutical Sciences, Lippincott Williams & Wilkins; 21 edition (May 1, 2005). Examples of immediate release systems include, but are not limited to, conventional tablets or capsules, or solutions. Examples of modified release systems include, but are not limited to, coated pellets, tablets or capsules; multiple unit or multiparticulate systems in the form of microparticles or nonoparticles, microspheres or pellets comprising the active agent; formulations comprising dispersions or solid solutions of active compound in a matrix, which may be in the form of a wax, gum, fat, or polymer; devices, in which drug is attached to an ion exchange resin, which provides for gradual release of drug by way of influence of other ions present in the gastrointestinal tract, for example, the acid environment of the stomach; devices, such as osmotic pumps, in which release rate of drug is controlled by way of its chemical potential; systems in which drug is released by diffusion through membranes, including multilayer systems, and so forth.

In some embodiments, the instant composition may be delivered from a system which may provide at least a part of the dose by a modified release system and another part by the immediate release system. Such systems may be constructed according to different principles, such as by single dose layered pellets or tablets, by multiple dose layered pellets or tablets, or by two or more different fractions of single or multiple dose layered pellets or tablets, optionally in combination with pellets or tablets having instant release.

In one embodiment, the carboxylic acid losartan dosage form is suitable for parenteral administration. Parenteral administration is generally characterized by injection, either subcutaneously, intramuscularly, or intravenously. Thus, compositions for intravenous administration comprise a solution of carboxylic acid losartan dissolved or suspended in an acceptable carrier. Injectables can be prepared as liquid solutions or suspensions, solid forms suitable for solution or suspension in liquid prior to injection, or as emulsions. Suitable excipients include, for example, water, buffered water, saline, dextrose, glycerol, ethanol, and the like. These compositions will be sterilized by conventional sterilization techniques, such as sterile filtration. The resulting solutions are packaged for use as is or lyophilized, the lyophilized preparation being combined with a sterile solution prior to administration. In addition, if desired, the pharmaceutical compositions to be administered may also contain minor amounts of non-toxic auxiliary substances, such as wetting or emulsifying agents, pH buffering agents and the like, such as for example, sodium acetate, sorbitan monolaurate, triethanolamine oleate, and combinations comprising one or more of the foregoing agents.

Besides the active ingredient, the dosage forms may include excipients (or pharmaceutically acceptable carriers) such as solvents, binders, fillers, disintegrants, lubricants, suspending agents, surfactants, viscosity increasing agents, buffering agents, antimicrobial agents, among others. The acceptable excipients and methods for making various dosages are known and may be found, for example, in Remington's Pharmaceutical Sciences, Lippincott Williams & Wilkins, 21^{st} edition, (May 1, 2005).

In several embodiments, a carboxylic acid losartan dosage form or its combination is suitable for oral administration. Oral administration may involve swallowing, so that the compound enters the gastrointestinal tract, or buccal or sublingual administration may be employed by which the compound enters the blood stream directly from the mouth. Formulations suitable for oral administration include solid formulations such as tablets; capsules containing particulates, liquids, or powders; lozenges (including liquid-filled); chews; multi- and nano-particulates; gels; solid solution; liposome; films; sprays; and liquid formulations.

In one embodiment, a carboxylic acid losartan dosage form or its combination comprises a buffering agent. Suitable buffering agents include sodium carbonate sodium bicarbonate, potassium carbonate, potassium bicarbonate, sodium phosphate, sodium biphosphate, potassium phosphate monobasic, potassium phosphate dibasic, organic bases, amines, and combinations comprising one or more of the foregoing buffering agents.

In one embodiment, a carboxylic acid losartan dosage form or its combination is a delayed-release dosage form. “Delayed-release” means that there is a time-delay before significant plasma levels of the active agent are achieved. A delayed-release formulation of an active agent can avoid an initial burst of the active agent, or can be formulated so that release of the active agent in the stomach is avoided and absorption is affected in the small intestine.

In one embodiment, delayed-release tablets comprise a core, a first coating and optionally a second coating. The core includes the carboxylic acid losartan, and excipients, such as a lubricant, and a binder and/or a filler, and optionally a glidant as well as other excipients.

Suitable lubricants include, for example, stearic acid, magnesium stearate, glyceryl behenate, talc, mineral oil (in PEG), and combinations comprising one or more of the foregoing lubricants. Suitable binders include, for example, water-soluble polymers, such as modified starch, gelatin, polyvinylpyrrolidone, polyvinyl alcohol, and combinations comprising one or more of the foregoing lubricants. Suitable fillers include, for example, lactose, microcrystalline cellulose, and the like. An example of a glidant is silicon dioxide (AEROSIL®, Degussa).

The core comprises, for example, by dry weight, about 0.1 to about 50 wt % carboxylic acid losartan or a pharmaceutically acceptable salt thereof, about 0.5 to about 10 wt % lubricant, and about 2 to about 98 wt % binder or filler.

In one embodiment, the first coating comprises a semi-permeable coating to achieve delayed-release of the carboxylic acid losartan. The first coating comprises, for example, a water-insoluble, film-forming polymer, together with a plasticizer and a water-soluble polymer. Suitable water-insoluble, film-forming polymers include, for example, cellulose ethers, such as ethylcellulose; cellulose esters, such as cellulose acetate; polyvinylalcohol; and combinations comprising one or more of the foregoing water-insoluble, film-forming polymers. A suitable water-insoluble film-forming polymer is ethylcellulose (available from Dow Chemical under the trade name ETHOCEL®). Suitable water-soluble polymers include polyvinylpyrrolidone. Other excipients are optionally present in the first coating, such as, for example, acrylic acid derivatives (e.g., EUDRAGIT®, Rohm Pharma, Degussa), pigments, etc.

The first coating contains about 20 to about 85 wt % water-insoluble, polymer (e.g., ethylcellulose), about 10 to about 75 wt % water-soluble polymer (e.g., polyvinylpyrrolidone), and about 5 to about 30 wt % plasticizer. The relative proportions of ingredients, notably the ratio of water-insoluble, film-forming polymer to water-soluble polymer, can be varied depending on the release profile to be obtained (where a more delayed-release is generally obtained with a higher amount of water-insoluble, film-forming polymer).

The weight ratio of first coating to tablet core is about 1:30 to about 3:10, specifically about 1:10.

The optional second coating is designed to protect the coated tablet core from coming into contact with gastric juice, thereby preventing a food effect. The second coating comprises, for example, an enteric polymer of the methacrylic type and optionally a plasticizer. The second coating comprises, for example, about 40 to about 95 wt % enteric polymer (e.g., EUDRAGIT® L30D-55) and about 5 to about 60 wt % plasticizer (e.g., triethyl citrate, polyethylene glycol). The relative proportions of ingredients, notably the ratio of methacrylic polymer to plasticizer can be varied according to a methods known to those of skill in the art of pharmaceutical formulation.

An exemplary process for preparing a delayed-release dosage form or its combination of the carboxylic acid losartan comprises manufacturing a core by, for example, wet or dry granulation techniques. Alternatively, the carboxylic acid losartan and lubricant may be mixed in a granulator and heated to the melting point of the lubricant to form granules. This mixture is then mixed with a suitable filler and compressed into tablets. Alternatively, the carboxylic acid losartan and a lubricant (e.g., mineral oil in PEG) are mixed in a granulator, e.g., a fluidized bed granulator and then into tablets. Tablets are formed by standard techniques, e.g., on a (rotary) press (for example KILIAN®) fitted with suitable punches. The resulting tablets are hereinafter referred as tablet cores.

An exemplary coating process follows. Ethylcellulose and polyethylene glycol (e.g., PEG 1450) are dissolved in a solvent such as ethanol; polyvinylpyrrolidone is then added. The resulting solution is sprayed onto the tablet cores, using a coating pan or a fluidized bed apparatus.

An exemplary process for applying the second coating follows. Triethyl citrate and polyethylene glycol (e.g., PEG 1450) are dissolved in a solvent such as water; a methacrylic polymer dispersion is then added. Silicon dioxide is optionally added as a suspension. The resulting solution is sprayed onto the coated tablet cores, using a coating pan or a fluidized bed apparatus.

The weight ratio of the second coating to coated tablet core is about 1:30 to about 3:10, specifically about 1:10.

An exemplary delayed-release dosage form or its combination comprises a core containing carboxylic acid losartan, polyvinylalcohol and glyceryl behenate; a first coating of ethylcellulose, polyvinylpyrrolidone, and polyethylene glycol; and a second coating of methacrylic acid co-polymer type C, triethyl citrate, polyethylene glycol, and optionally containing silicon dioxide.

In another embodiment, the carboxylic acid losartan dosage form or its combination is a sustained- or extended-release dosage form. By “sustained-release” or “extended-release” are meant to include formulations designed to release the active agent at such a rate that blood (e.g., plasma) levels are maintained within a therapeutic range but below toxic levels for at least about 8 hours, specifically at least about 12 hours after administration at steady-state. The term “steady-state” means that a plateau plasma level for a given active agent has been achieved and which is maintained with subsequent doses of the drug at a level which is at or above the minimum effective therapeutic level and is below the minimum toxic plasma level for a given active agent. With regard to dissolution profiles, the first and second dissolution profiles (e.g., in the stomach and in the intestines) should each be equal to or greater than the minimum dissolution required to provide substantially equivalent bioavailability to a capsule, tablet or liquid containing the at least one active ingredient in an immediate-release form.

In one embodiment, a sustained-release carboxylic acid losartan dosage form has a reduced Cmax compared to an immediate-release formulation comprising either carboxylic acid losartan. The sustained-release carboxylic acid losartan dosage form can maintain bioavailability and minimum effective concentration substantially equivalent to that of the immediate release composition of carboxylic acid losartan upon multiple dosing. In one embodiment, a sustained-release dosage form comprising carboxylic acid losartan, when ingested orally, has a lower fluctuation index in the plasma than an immediate release composition of carboxylic acid losartan while maintaining bioavailability substantially equivalent to that of the immediate release composition of carboxylic acid losartan. As used herein, the fluctuation index or “Degree of Fluctuation (DFL)” as used herein, is expressed as: DFL=(Cmax−Cmin)/Cavg.

A sustained-release form is a form suitable for providing controlled-release of the carboxylic acid losartan over a sustained period of time (e.g., 12 hours, 24 hours). In one embodiment, sustained-release dosage forms of carboxylic acid losartan release the carboxylic acid losartan at a rate independent of pH, for example, about pH 1.2 to about 7.5. Alternatively, sustained-release dosage forms release carboxylic acid losartan at a rate dependent upon pH, for example a lower rate of release at pH 1.2 and a higher rate of release at pH 7.5. Typically, the sustained-release form avoids “dose dumping” upon oral administration. The sustained-release oral dosage form can be formulated to provide for an increased duration of carboxylic acid losartan action allowing once-daily dosing.

A sustained-release dosage form comprises a release-retarding material in the form of, for example, a matrix or a coating. The carboxylic acid losartan in sustained-release form comprises, for example, a particle of the carboxylic acid losartan that is combined with a release-retarding material. The release-retarding material is a material that permits release of the carboxylic acid losartan at a sustained rate in an aqueous medium. The release-retarding material is selectively chosen so as to achieve, in combination with the other stated properties, a desired in vitro release rate.

Release-retarding materials include hydrophilic and/or hydrophobic polymers. Release-retarding materials include, for example acrylic polymers, alkylcelluloses, shellac, zein, hydrogenated vegetable oil, hydrogenated castor oil, and combinations comprising one or more of the foregoing materials. The oral dosage form contains about 1 wt % to about 80 wt % of the release-retarding material. Suitable acrylic polymers include, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, and combinations comprising one or more of the foregoing polymers. Suitable acrylic polymers include methacrylate copolymers described in NF XXIV as fully polymerized copolymers of acrylic and methacrylic acid esters with a low content of quaternary ammonium groups.

Suitable alkylcelluloses include, for example, ethylcellulose. Those skilled in the art will appreciate that other cellulosic polymers, including other alkyl cellulosic polymers, can be substituted for part or all of the ethylcellulose.

Other suitable hydrophobic materials are water-insoluble with more or less pronounced hydrophobic trends. The hydrophobic material has, for example, a melting point of about 30° C. to about 200° C., more specifically about 45° C. to about 9° C. Exemplary hydrophobic materials include natural or synthetic waxes, fatty alcohols (such as lauryl, myristyl, stearyl, cetyl or preferably cetostearyl alcohol), fatty acids, including fatty acid esters, fatty acid glycerides (mono-, di-, and tri-glycerides), hydrogenated fats, hydrocarbons, normal waxes, stearic acid, stearyl alcohol, hydrophobic and hydrophilic materials having hydrocarbon backbones, and combinations comprising one or more of the foregoing materials. Suitable waxes include beeswax, glycowax, castor wax, carnauba wax and wax-like substances, e.g., materials normally solid at room temperature and having a melting point of about 30° C. to about 100° C., and combinations comprising one or more of the foregoing waxes.

In other embodiments, the release-retarding material comprises digestible, long chain (e.g., C8-C50, specifically C12-C40), substituted or unsubstituted hydrocarbons, such as fatty acids, fatty alcohols, glyceryl esters of fatty acids, mineral and vegetable oils, waxes, and combinations comprising one or more of the foregoing materials. Hydrocarbons having a melting point of between about 25° C. and about 90° C. may be employed. Of these long chain hydrocarbon materials, fatty (aliphatic) alcohols are preferred. The oral dosage form comprises up to about 60 wt % of at least one digestible, long chain hydrocarbon.

Further, the sustained-release matrix comprises up to 60 wt % of at least one polyalkylene glycol.

Alternatively, the release-retarding material comprises polylactic acid, polyglycolic acid, or a co-polymer of lactic and glycolic acid.

Release-modifying agents, which affect the release properties of the release-retarding material, are optionally employed. The release-modifying agents function, for example, as pore-formers. The pore former can be organic or inorganic, and includes materials that can be dissolved, extracted or leached from the coating in the environment of use. Suitable pore-formers include one or more hydrophilic polymers, such as hydroxypropylmethyl cellulose, hydroxypropylcellulose, polycarbonates comprised of linear polyesters of carbonic acid in which carbonate groups reoccur in the polymer chain. Alternatively, suitable pore formers include small molecules such as lactose or metal stearates, and combinations comprising one or more of the foregoing release-modifying agents.

The release-retarding material also optionally includes other additives such as an erosion-promoting agent (e.g., starch and gums); and/or a semi-permeable polymer. In addition to the above ingredients, a sustained-release dosage form optionally also contains suitable quantities of other materials, e.g., diluents, lubricants, binders, granulating aids, colorants, flavorants and glidants that are conventional in the pharmaceutical art. The release-retarding material optionally includes an exit means comprising at least one passageway, orifice, or the like. The passageway can have a suitable shape, such as round, triangular, square, elliptical, irregular, etc.

The sustained-release dosage form comprising carboxylic acid losartan and a release-retarding material is prepared by a suitable technique for preparing carboxylic acid losartan as described in detail below. The carboxylic acid losartan and release-retarding material are, for example, prepared by wet granulation techniques, melt extrusion techniques, and the like. To obtain a sustained-release dosage form, it may be advantageous to incorporate an additional hydrophobic material.

The carboxylic acid losartan in sustained-release form optionally includes a plurality of substrates comprising the carboxylic acid losartan, which substrates are coated with a sustained-release coating comprising a release-retarding material. The sustained-release preparations may thus be made in conjunction with a multiparticulate system, such as beads, ion-exchange resin beads, spheroids, microspheres, seeds, pellets, granules, and other multiparticulate systems in order to obtain a desired sustained-release of the carboxylic acid losartan. The multiparticulate system is presented in a capsule or other suitable unit dosage form.

In certain cases, more than one multiparticulate system can be employed, each exhibiting different characteristics, such as pH dependence of release, time for release in various media (e.g., acid, base, simulated intestinal fluid), release in vivo, size, and composition.

In some cases, a spheronizing agent, together with the carboxylic acid losartan is spheronized to form spheroids. Microcrystalline cellulose and hydrous lactose impalpable are examples of spheronizing agents. Additionally (or alternatively), the spheroids contain a water insoluble polymer, suitably an acrylic polymer, an acrylic copolymer, such as a methacrylic acid-ethyl acrylate copolymer, or ethyl cellulose. In this formulation, the sustained-release coating will generally include a water insoluble material such as a wax, either alone or in admixture with a fatty alcohol, or shellac or zein.

Spheroids or beads, coated with carboxylic acid losartan are prepared, for example, by dissolving or dispersing the carboxylic acid losartan in a solvent such as water and then spraying the solution onto a substrate, for example, sugar spheres NF, 18/20 mesh, using a Wurster insert. Optionally, additional ingredients are also added prior to coating the beads in order to assist the carboxylic acid losartan binding to the substrates, and/or to color the resulting beads, etc. The resulting substrate- carboxylic acid losartan may optionally be overcoated with a barrier material, to separate the carboxylic acid losartan from the next coat of material, e.g., release-retarding material. Specifically, the barrier material is a material comprising hydroxypropylmethylcellulose. However, a film-former known in the art may be used. Preferably, the barrier material does not affect the dissolution rate of the final product.

To obtain a sustained-release of the carboxylic acid losartan in a manner sufficient to provide the desired effect for the sustained durations, the substrate comprising the carboxylic acid losartan is coated with an amount of release-retarding material sufficient to obtain a weight gain level from about 2 to about 30 wt %, although the coat can be greater or lesser depending upon the physical properties of the carboxylic acid losartan and the desired release rate, among other things. Moreover, there can be more than one release-retarding material used in the coat, as well as various other pharmaceutical excipients.

In one embodiment, the release-retarding material is in the form of a film coating comprising a dispersion of a hydrophobic polymer. Solvents typically used for application of the release-retarding coating include pharmaceutically acceptable solvents, such as water, methanol, ethanol, methylene chloride, and combinations comprising one or more of the foregoing solvents.

In addition, the sustained-release profile of carboxylic acid losartan release in the formulations (either in vivo or in vitro) can be altered, for example, by using more than one release-retarding material, varying the thickness of the release-retarding material, changing the particular release-retarding material used, altering the relative amounts of release-retarding material, altering the manner in which the plasticizer is added (e.g., when the sustained-release coating is derived from an aqueous dispersion of hydrophobic polymer), by varying the amount of plasticizer relative to retardant material, by the inclusion of additional ingredients or excipients, by altering the method of manufacture, etc.

In addition to or instead of being present in a matrix, the release-retarding agent can be in the form of a coating. Optionally, the dosage forms can be coated, or a gelatin capsule can be further coated, with a sustained-release coating such as the sustained-release coatings described herein. Such coatings are particularly useful when the subunit comprises the carboxylic acid losartan in releasable form, but not in sustained-release form. Suitable coatings include a sufficient amount of a hydrophobic material to obtain a weight gain level from about 2 to about 30 wt %, although the overcoat can be greater upon the physical properties of the particular the active agent and the desired release rate, among other things.

The sustained-release formulations preferably slowly release the carboxylic acid losartan, e.g., when ingested and exposed to gastric fluids, and then to intestinal fluids. The sustained-release profile of the formulations can be altered, for example, by varying the amount of retardant, e.g., hydrophobic material, by varying the amount of plasticizer relative to hydrophobic material, by the inclusion of additional ingredients or excipients, by altering the method of manufacture, etc.

In one embodiment, a carboxylic acid losartan dosage form or its combination is a controlled-release matrix formulation. An exemplary controlled-release formulation is one in which the carboxylic acid losartan is dispersed in a polymeric matrix that is water-swellable rather than merely hydrophilic, that has an erosion rate that is substantially slower than its swelling rate, and that releases the carboxylic acid losartan primarily by diffusion. The rate of diffusion of the carboxylic acid losartan out of the matrix can be slowed by increasing the carboxylic acid losartan particle size, by the choice of polymer used in the matrix, and/or by the choice of molecular weight of the polymer. The matrix is a relatively high molecular weight polymer that swells upon ingestion, preferably to a size that is at least about twice its unswelled volume, and that promotes gastric retention during the fed mode. Upon swelling, the matrix may also convert over a prolonged period of time from a glassy polymer to a polymer that is rubbery in consistency, or from a crystalline polymer to a rubbery one. The penetrating fluid then causes release of the carboxylic acid losartan in a gradual and prolonged manner by the process of solution diffusion, i.e., dissolution of the carboxylic acid losartan in the penetrating fluid and diffusion of the dissolved carboxylic acid losartan back out of the matrix. The matrix itself is solid prior to administration and, once administered, remains undissolved in (i.e., is not eroded by) the gastric fluid for a period of time sufficient to permit substantially all of the carboxylic acid losartan to be released by the solution diffusion process during the fed mode. By substantially all, it is meant greater than or equal to about 90 wt %, preferably greater than or equal to about 95 wt % of the carboxylic acid losartan or pharmaceutically acceptable salt thereof is released. The rate limiting factor in the release of the carboxylic acid losartan may be therefore controlled diffusion of the carboxylic acid losartan from the matrix rather than erosion, dissolving or chemical decomposition of the matrix.

For carboxylic acid losartan, the swelling of the polymeric matrix thus achieves two objectives—(i) the tablet swells to a size large enough to cause it to be retained in the stomach during the fed mode, and (ii) it retards the rate of diffusion of the carboxylic acid losartan long enough to provide multi-hour, controlled delivery of the carboxylic acid losartan into the stomach. The water-swellable polymer forming the matrix is a polymer that is non-toxic, that swells in a dimensionally unrestricted manner upon imbibition of water, and that provides for sustained-release of an incorporated active agent. Examples of suitable polymers include, for example, cellulose polymers and their derivatives (such as for example, hydroxyethylcellulose, hydroxypropylcellulose, carboxymethylcellulose, and microcrystalline cellulose, polysaccharides and their derivatives, polyalkylene oxides, polyethylene glycols, chitosan, poly(vinyl alcohol), xanthan gum, maleic anhydride copolymers, poly(vinyl pyrrolidone), starch and starch-based polymers, poly (2-ethyl-2-oxazoline), poly(ethyleneimine), polyurethane hydrogels, crosslinked polyacrylic acids and their derivatives, and combinations comprising one or more of the foregoing polymers. Further examples are copolymers of the polymers listed in the preceding sentence, including block copolymers and grafted polymers. Specific examples of copolymers are PLURONIC® and TECTRONIC®, which are polyethylene oxide-polypropylene oxide block copolymers available from BASF Corporation, Chemicals Div., Wyandotte, Mich., USA.

The terms “cellulose” and “cellulosic” denote a linear polymer of anhydroglucose. Cellulosic polymers include, for example, alkyl-substituted cellulosic polymers that ultimately dissolve in the gastrointestinal (GI) tract in a predictably delayed manner. Alkyl-substituted cellulose derivatives may be those substituted with alkyl groups of 1 to 3 carbon atoms each. Specific examples are methylcellulose, hydroxymethylcellulose, hydroxyethylcellulose, hydroxypropylcellulose, hydroxypropylmethylcellulose, and carboxymethylcellulose. In terms of their viscosities, one class of suitable alkyl-substituted celluloses includes those whose viscosity is about 100 to about 110,000 centipoise as a 2% aqueous solution at 20° C. Another class includes those whose viscosity is about 1,000 to about 4,000 centipoise as a 1% aqueous solution at 20° C. Exemplary alkyl-substituted celluloses are hydroxyethylcellulose and hydroxypropylmethylcellulose. A specific example of a hydroxyethylcellulose is NATRASOL® 250HX NF (National Formulary), available from Aqualon Company, Wilmington, Del., USA.

Suitable polyalkylene oxides are those having the properties described above for alkyl-substituted cellulose polymers. An example of a polyalkylene oxide is poly(ethylene oxide), which term is used herein to denote a linear polymer of unsubstituted ethylene oxide. Poly(ethylene oxide) polymers having molecular weights of about 4,000,000 and higher are particularly suitable. More preferred are those with molecular weights of about 4,500,000 to about 10,000,000, and even more preferred are polymers with molecular weights of about 5,000,000 to about 8,000,000. Preferred poly(ethylene oxide)s are those with a weight-average molecular weight of about 1×105 to about 1×107, and preferably within the range of about 9×105 to about 8×106. Poly(ethylene oxide)s are often characterized by their viscosity in solution. A preferred viscosity is about 50 to about 2,000,000 centipoise for a 2% aqueous solution at 20° C. Two specific example of poly(ethylene oxide)s are POLYOX® NF, grade WSR Coagulant, molecular weight 5 million, and grade WSR 303, molecular weight 7 million, both available from Dow.

Polysaccharide gums, both natural and modified (semi-synthetic) can be used. Examples are dextran, xanthan gum, gellan gum, welan gum and rhamsan gum.

Crosslinked polyacrylic acids of greatest utility are those whose properties are the same as those described above for alkyl-substituted cellulose and polyalkylene oxide polymers. Preferred crosslinked polyacrylic acids are those with a viscosity of about 4,000 to about 40,000 centipoise for a 1% aqueous solution at 25° C. Three specific examples are CARBOPOL® NF grades 971P, 974P and 934P (BFGoodrich Co., Specialty Polymers and Chemicals Div., Cleveland, Ohio, USA).

The hydrophilicity and water swellability of these polymers cause the carboxylic acid losartan -containing matrices to swell in size in the gastric cavity due to ingress of water in order to achieve a size that will be retained in the stomach when introduced during the fed mode. These qualities also cause the matrices to become slippery, which provides resistance to peristalsis and further promotes their retention in the stomach. The release rate of carboxylic acid losartan from the matrix is primarily dependent upon the rate of water imbibition and the rate at which the carboxylic acid losartan dissolves and diffuses from the swollen polymer, which in turn is related to the solubility and dissolution rate of the carboxylic acid losartan, the carboxylic acid losartan particle size and the carboxylic acid losartan concentration in the matrix. Also, because these polymers dissolve very slowly in gastric fluid, the matrix maintains its physical integrity over at least a substantial period of time, in many cases at least 90%, and preferably over 100% of the dosing period. The particles will then slowly dissolve or decompose. Complete dissolution or decomposition may not occur until 24 hours or more after the intended dosing period ceases, although in most cases, complete dissolution or decomposition will occur within 10 to 24 hours after the dosing period.

The dosage forms optionally include additives that impart a small degree of hydrophobic character, to further retard the release rate of the carboxylic acid losartan into the gastric fluid. One example of such a release rate retardant is glyceryl monostearate. Other examples are fatty acids and salts of fatty acids, one example of which is sodium myristate. The quantities of these additives when present can vary; and in most cases, the weight ratio of additive to carboxylic acid losartan will be about 1:20 to about 1:1, and preferably about 1:8 to about 1:2.

The amount of polymer relative to the carboxylic acid losartan can vary, depending on the carboxylic acid losartan release rate desired and on the polymer, its molecular weight, and excipients that may be present in the formulation. The amount of polymer should be sufficient however to retain at least about 40% of the carboxylic acid losartan within the matrix one hour after ingestion, or immersion in simulated gastric fluid. As used herein, simulated gastric fluid refers to 0.1 N hydrochloric acid. Specifically, the amount of polymer is such that at least about 50% of the carboxylic acid losartan remains in the matrix one hour after ingestion, or immersion in simulated gastric fluid. More specifically, at least about 60%, and most preferably at least about 80%, of the carboxylic acid losartan remains in the matrix one hour after ingestion, or immersion in simulated gastric fluid. In all cases, however, the carboxylic acid losartan will be substantially all released from the matrix within about ten hours, and preferably within about eight hours, after ingestion or immersion in simulated gastric fluid, and the polymeric matrix will remain substantially intact until all of the carboxylic acid losartan is released. The term “substantially intact” is used herein to denote a polymeric matrix in which the polymer portion substantially retains its size and shape without deterioration due to becoming solubilized in the gastric fluid or due to breakage into fragments or small particles.

The water-swellable polymers can be used individually or in combination. Certain combinations will often provide a more controlled-release of the carboxylic acid losartan than their components when used individually. An exemplary combination is cellulose-based polymers combined with gums, such as hydroxyethyl cellulose or hydroxypropyl cellulose combined with xanthan gum. Another example is poly(ethylene oxide) combined with xanthan gum.

The benefits of this dosage form will be achieved over a wide range of carboxylic acid losartan loadings, with the weight ratio of carboxylic acid losartan to polymer of 0.01:99.99 to about 80:20. Preferred loadings (expressed in terms of the weight percent of carboxylic acid losartan relative to total of active agent and polymer) are about 0.1% to about 10%, more preferably about 0.1% to about 5%, and most preferably in certain cases about 0.1% to about 3.5%.

The dosage forms may find their greatest utility when administered to a subject who is in the digestive state (also referred to as the postprandial or “fed” mode). The postprandial mode is distinguishable from the interdigestive (or “fasting”) mode by their distinct patterns of gastroduodenal motor activity, which determine the gastric retention or gastric transit time of the stomach contents.

In one embodiment, a carboxylic acid losartan dosage form is a pulsed-release dosage form. A “pulsed-release” formulation comprises a combination of immediate-release, sustained-release, and/or delayed-release formulations in the same dosage form. A “semi-delayed-release” formulation is a pulsed-released formulation in which a moderate dosage is provided immediately after administration and a further dosage some hours after administration. The immediate-release portion is sometimes referred to as a loading dose.

An exemplary pulsed-release dosage form provides at least a part of the dose with a pulsed delayed-release of the carboxylic acid losartan and another part of the formulation with rapid or immediate-release. The immediate-release and delayed-release dosage forms contain the same or different amounts of carboxylic acid losartan. In some embodiments, the delayed-release dosage form has a higher concentration of carboxylic acid losartan than the immediate-release dosage form. The immediate and pulsed delayed-release of the drug can be achieved according to different principles, such as by single dose layered pellets or tablets, by multiple dose layered pellets or tablets, or by two or more different fractions of single or multiple dose layered pellets or tablets, optionally in combination with pellets or tablets having instant release. Multiple dose layered pellets may be filled into a capsule or together with tablet excipients compressed into a multiple unit tablet. Alternatively, a multiple dose layered tablet may be prepared.

In one embodiment, single dose layered pellets or tablets give one single delayed-release pulse of the carboxylic acid losartan. The single dose layered pellets or tablets comprise, for example, a core material, optionally layered on a seed/sphere, the core material comprising the carboxylic acid losartan together with a water swellable substance; a surrounding lag time controlling layer, and an outer coating layer positioned to cover the lag time controlling layer. Alternatively, the layered pellets or tablets comprise a core material comprising the carboxylic acid losartan; a surrounding layer comprising a water swellable substance; a surrounding lag time controlling layer; and an outer coating layer positioned to cover the lag time controlling layer.

In one embodiment, multiple dose layered pellets or tablets giving two or more delayed-release pulses of the carboxylic acid losartan comprise a core material, optionally layered on a seed/sphere comprising the carboxylic acid losartan and a water swellable substance, a surrounding lag time controlling layer, a layer comprising the carboxylic acid losartan optionally together with a water swellable substance; optionally a separating layer which is water-soluble or in water rapidly disintegrating; and an outer coating layer. Alternatively, multiple dose layered pellets or tablets comprise a core material, optionally layered on a seed/sphere, comprising the carboxylic acid losartan; a surrounding layer comprising a water swellable substance; a surrounding lag time controlling layer; a layer comprising the carboxylic acid losartan; optionally a separating layer; and an outer coating layer.

The core material comprising the carboxylic acid losartan is prepared either by coating or layering the carboxylic acid losartan onto a seed, such as for instance sugar spheres, or by extrusion/spheronization of a mixture comprising the carboxylic acid losartan and pharmaceutically acceptable excipients. It is also possible to prepare the core material by using tablet technology, i.e., compression of carboxylic acid losartan granules and optionally pharmaceutically acceptable excipients into a tablet core. For pellets of the two types, i.e., single or multiple dose pellets, which have the carboxylic acid losartan deposited onto a seed/sphere by layering, it is also possible to have an optional layer comprising a water swellable substance beneath the carboxylic acid losartan—containing layer in the core material. The seeds/spheres are typically water insoluble and comprise different oxides, celluloses, organic polymers and other materials, alone or in mixtures, or be water soluble and comprise different inorganic salts, sugars and other materials, alone or in mixtures. Further, the seeds/spheres may comprise the carboxylic acid losartan in the form of crystals, agglomerates, compacts etc. The size of the seeds is about 0.1 to about 2 mm. Before the seeds are layered, the carboxylic acid losartan is optionally mixed with further components to obtain suitable handling and processing properties and a suitable concentration of the carboxylic acid losartan in the final mixture.

Optionally an osmotic agent is placed in the core material. Such an osmotic agent is water soluble and will provide an osmotic pressure in the tablet. Examples of osmotic agents are magnesium sulfate, sodium chloride, lithium chloride, potassium chloride, potassium sulfate, sodium carbonate, lithium sulfate, calcium bicarbonate, sodium sulfate, calcium lactate, urea, magnesium succinate, sucrose, and combinations comprising one or more of the foregoing osmotic agents.

Water swellable substances suitable for the pellet dosage forms are compounds which are able to expand when they are exposed to an aqueous solution, such as gastro-intestinal fluid. One or more water swellable substances may be present in the core material together with the carboxylic acid losartan and optionally pharmaceutically acceptable excipient(s). Alternatively, one or more water swellable substances are included in a swelling layer applied onto the core material. As a further alternative, swellable substances(s) they may also be present in an optional swelling layer situated beneath the drug containing layer, if a layered seed or sphere is used as the core material.

The amount of water swellable substance(s) in the swelling layer or in the core to material is chosen in such a way that the core material or the swelling layer in contact with an aqueous solution, such as gastrointestinal fluid, will expand to such a degree that the surrounding lag-time controlling membrane ruptures. A water swellable substance may also be included in the drug comprising layer of the multiple layered pellets or tablets to increase dissolution rate of the drug fraction.

Suitable water swellable substances include, for example, low-substituted hydroxypropyl cellulose, e.g., L-HPC; cross-linked polyvinyl pyrrolidone (PVP-XL), e.g., Kollidon® CL and Polyplasdone® XL; cross-linked sodium carboxymethylcellulose, e.g., Ac-di-sol®, Primellose®; sodium starch glycolate, e.g., Primojel®; sodium carboxymethylcellulose, e.g., Nymcel ZSB10®; sodium carboxymethyl starch, e.g., Explotab®; ion-exchange resins, e.g., Dowex® or Amberlite®; microcrystalline cellulose, e.g., Avicel®; starches and pregelatinized starch, e.g., Starch 1500®, Sepistab ST200®; formalin-casein, e.g., Plas-Vita®, and combinations comprising one or more of the foregoing water swellable substances.

The core optionally comprises an absorption enhancer. Suitable absorption enhancers include, for example, a fatty acid, a surfactant, a chelating agent, a bile salt, and combinations comprising one or more of the foregoing absorption enhancers. Specific examples of absorption enhancers are fatty acids such as capric acid, oleic acid and their monoglycerides, surfactants such as sodium lauryl sulfate, sodium taurocholate and polysorbate 80, chelating agents such as citric acid, phytic acid, ethylenediamine tetraacetic acid (EDTA) and ethylene glycol-bis(β-aminoethyl ether)-N,N,N,N-tetraacetic acid (EGTA). The core comprises about 0 to about 20 wt % of the absorption enhancer based on the total weight of the core and more specifically about 2 wt % to about 10 wt % of the total weight of the core.

In one embodiment, the pulsed-release dosage form comprises a lag time controlling layer. A lag time controlling layer is a semipermeable membrane comprising a water resistant polymer that is semipermeable for an aqueous solution, such as gastro-intestinal fluid. Suitable polymers are cellulose acetate, ethylcellulose, polyvinyl acetate, cellulose acetate butyrate, cellulose acetate propionate, acrylic acid copolymers, such as Eudragit® RS or RL, and combinations comprising one or more of the foregoing polymers. The layer optionally comprises pore forming agents, such as a water soluble substance, e.g., sucrose, salt; or a water soluble polymer e.g., polyethylene glycol. Also pharmaceutically acceptable excipients, such as fillers and membrane strength influencing agents such as talc, aerosil, and sodium aluminum silicate, may be included.

The lag time controlling layer is typically positioned nearest the inner core material and is constructed in the form of a semipermeable membrane that will disrupt after a desired time after ingestion. A desired lag time may be adjusted by the composition and thickness of the layer. The amount of substances forming such a disrupting semipermeable membrane, i.e., a lag time controlling layer, is about 0.5 to about 25 wt % of the weight of the core material including swelling substances or a swelling layer, preferably about 2 to about 20 wt %.

In one embodiment, the lag time controlling layer comprises a mixture of ethylcellulose and talc. The mixture contains 10 to 80 wt % w/w of talc.

Before applying the outer coating layer onto the layered pellets or tablets, they are optionally covered with one or more separating layers comprising excipients. The separating layer separates the composition of the layered pellets or tablets from the outer enteric coating layer. Suitable materials for the optional separating layer are pharmaceutically acceptable compounds such as, for example, sugar, polyethylene glycol, polyvinyl pyrrolidone, polyvinyl alcohol, polyvinyl acetate, hydroxypropyl cellulose, methylcellulose, ethylcellulose, hydroxypropyl methylcellulose, carboxymethylcellulose sodium and others, and combinations comprising one or more of the foregoing materials. Other additives may also be included into the separating layer.

When the optional separating layer is applied to the layered pellets or tablets, it constitutes a variable thickness. The maximum thickness of the optional separating layer is limited only by processing conditions. The separating layer may serve as a diffusion barrier and may act as a pH-buffering zone. The optional separating layer is employed to improve the chemical stability of the carboxylic acid losartan and/or the physical properties of the dosage form.

Finally the layered pellets or tablets are covered by one or more outer coating layers by using a suitable coating technique. The outer coating layer material is dispersed or dissolved in either water or in suitable organic solvents. Suitable polymers for the coating material include methacrylic acid copolymers, cellulose acetate phthalate, hydroxypropyl methylcellulose phthalate, hydroxypropyl methylcellulose acetate succinate, polyvinyl acetate phthalate, cellulose acetate trimellitate, carboxymethyl ethylcellulose, shellac or other suitable coating layer polymer(s), and combinations comprising one or more of the foregoing polymers.

The applied polymer containing layers, specifically the outer coating layers, optionally contain pharmaceutically acceptable plasticizers to obtain desired mechanical properties.

In one embodiment, the carboxylic acid losartan dosage form is a wax formulation. A wax formulation is a solid dosage form comprising the carboxylic acid losartan or a pharmaceutically acceptable salt thereof, in a waxy matrix. The waxy matrix is prepared, for example, by hot melting a suitable wax material and using the melt to granulate the carboxylic acid losartan. The matrix material comprises the waxy material and the carboxylic acid losartan.

Suitable wax materials include, for example, an amorphous wax, an anionic wax, an anionic emulsifying wax, a bleached wax, a carnauba wax, a cetyl esters wax, a beeswax, a castor wax, a cationic emulsifying wax, a cetrimide emulsifying wax, an emulsifying wax, a glyceryl behenate, a microcrystalline wax, a nonionic wax, a nonionic emulsifying wax, a paraffin, a petroleum wax, a spermaceti wax, a white wax, a yellow wax, and combinations comprising one or more of the foregoing waxes. These and other suitable waxes are known to those of skill in the art. A cetyl esters wax, for example, typically has a molecular weight of about 470 to about 490 and is a mixture containing primarily esters of saturated fatty alcohols and saturated fatty acids. The wax material can comprise a carnauba wax, glyceryl behenates, castor wax, and combinations comprising one or more of the foregoing waxes. When the waxy material consists of carnauba wax and no other waxy material is used, the matrix is optionally coated with a functional coating. When the waxy material includes glyceryl behenates and carnauba wax, the matrix can be used without a coating, but may have either a cosmetic coating or a functional coating depending on the precise release profile and appearance desired.

The wax material is employed at about 16 wt % to about 35 wt %, specifically about 20 wt % to about 32 wt %, more specifically about 24 wt % to about 31 wt %, and most specifically about 28 wt % to about 29 wt % of the total weight of the matrix material. When a combination of wax is used, e.g., carnauba wax and glyceryl behenate, the component waxes can be used in a suitable ratio. Certain formulations include the wax material component from 100 to about 85 parts carnauba wax and from 0 to about 15 parts glyceryl behenate. In formulations that have a combination of carnauba wax and castor wax, for example, the wax component comprises, for example, about 100 to about 85 parts carnauba wax and 0 to about 15 parts castor wax. When carnauba wax, glyceryl behenate and castor wax are present, the carnauba wax comprises at least about 85 wt % of the waxy material and the balance of the waxy material is made up of a combination of glyceryl behenate and castor wax, in a suitable relative proportion.

Optionally, fatty acids and fatty acid soaps can be present in the wax dosage form. In some cases, the fatty acids and/or fatty acid soaps replace a portion of the wax or waxes. These optional fatty acids and fatty acid soaps include those that are generally used in the pharmaceutical industry as tableting lubricants, such as, for example, solid fatty acids (for example fatty acids having from about 16 to about 22 carbon atoms), and the alkaline earth metal salts thereof, particularly the magnesium and calcium salts, and combinations comprising one or more of the foregoing fatty acids. The fatty acid can be, for example, stearic acid. The optional fatty acids and fatty acid soaps, when present, are used in amounts of up to about 10 wt % of the total weight of the matrix material, or about 2.5 wt % to about 9 wt %, or about 2.7 wt % to about 8.6 wt %, or about 3 wt % to about 6 wt % of the total weight of the matrix material. An amount of up to about 2 wt % of the total core formulation of the optional fatty acid materials may be used as a blend with the melt granulate. Amounts of at least about 1% may be used in this fashion with the remainder being added to the waxes for melting and granulating the rosiglitazone.

To prepare the dosage form, the waxes are melted and used to granulate the carboxylic acid losartan. The granulate is allowed to cool and then milled to a proper size. Advantageously, the granulate is milled to an average particle size of about 75 microns to about 850 microns, specifically about 150 microns to about 425 microns. The milled granulate is optionally mixed with processing aids. The processing aids include, for example, hydrophobic colloidal silicon dioxide (such as CAB-O-SIL® M5). Hydrophobic silicon dioxide is typically employed in amounts of less than or equal to about 0.5 wt %, but individual formulations can be varied as required. The blend of the waxy granulate and the processing aids, if any, are compressed and then optionally coated.

The wax dosage form can include, for example, compressed coated or uncoated tablets, compressed pellets contained in capsules, or loose powder or powder filled capsules.

In one embodiment, a carboxylic acid losartan dosage form comprises a gum such as a polysaccharide gum to produce a sustained-release of the carboxylic acid losartan. Polysaccharide gums, both natural and modified (semi-synthetic) can be used. Examples are dextran, xanthan gum, gellan gum, welan gum and rhamsan gum. The gum is present, for example, in the form of a matrix comprising 10 to 80 wt % of the formulation, specifically 20 to 60 wt %.

In one embodiment, a carboxylic acid losartan dosage form is a bioadhesive dosage form designed to adhere to the epithelial surface of the stomach. Bioadhesive dosage forms comprise a bioadhesive polymer and additional excipients for the release of the carboxylic acid losartan to the stomach. Bioadhesive dosage forms can be tablets, capsules or granules comprising a bioadhesive polymer.

Suitable bioadhesive polymers include carbomer, polycarbophil, hydrodroxypropyl methyl cellulose, hydroxypropyl cellulose or admixtures thereof. Cationic bioadhesive polymers include acidic (high isoelectric point) gelatin; polygalactosamine; proteins (polyaminoacids) such as polylysine, polyomithine; polyquaternary compounds; prolamine; polyimine; diethylaminoethyldextran (DEAE); DEAE-imine; polyvinylpyridine; polythiodiethylaminomethylethylene (PTDAE); polyhistidine; DEAE-methacrylate; DEAE-acrylamide; poly-p-aminostyrene; polyoxethane; copolymethacrylates (e.g. copolymers of HPMA, N-(2-hydroxypropyl)-methacrylamide); Eudragit® RL; Eudragit® RS; polyamidoamines; cationic starches; DEAE-dextran; DEAE-cellulose; and combinations comprising one or more of the foregoing polymers.

In another embodiment, a carboxylic acid losartan dosage form dosage form is a chewable tablet containing the carboxylic acid losartan. A chewable tablet comprises a chewable base and optionally a sweetener. The chewable base comprises an excipient such as, for example, mannitol, sorbitol, lactose, or a combination comprising one or more of the foregoing excipients. The optional sweetener used in the chewable dosage form includes, for example, digestible sugars, sucrose, liquid glucose, sorbitol, dextrose, isomalt, liquid maltitol, aspartame, lactose, and combinations comprising one ore more of the foregoing sweeteners. In certain cases, the chewable base and the sweetener are the same component. The chewable base and optional sweetener comprise about 50 to about 90 wt % of the total weight of the dosage form.

The chewable dosage form optionally additionally contains preservatives, agents that prevent adhesion to oral cavity and crystallization of sugars, flavoring agents, souring agents, coloring agents, and combinations comprising one or more of the foregoing agents. Glycerin, lecithin, hydrogenated palm oil or glyceryl monostearate may be used as a protecting agent of crystallization of the sugars in an amount of about 0.04 to about 2.0 wt % of the total weight of the ingredients, to prevent adhesion to oral cavity and improve the soft property of the products. Additionally, isomalt or liquid maltitol may be used to enhance the chewing properties of the chewable dosage form.

A method of making a chewable dosage form of the carboxylic acid losartan is similar to the method used to make soft confectionary. The method generally involves the formation of a digestible sugar blend to which is added a frappe mixture. The boiled sugar blend is prepared, for example, from sugar and corn syrup blended in parts by weight ratio of 90:10 to 10:90. This blend is heated to temperatures above 250° F. to remove water and to form a molten mass. The frappe mixture is prepared from gelatin, egg albumen, milk proteins such as casein, and vegetable proteins such as soy protein, and the like which are added to a gelatin solution and rapidly mixed at ambient temperature to form an aerated sponge like mass. The frappe mixture is then added to the molten candy base and mixed until homogenous at temperatures between 150° F. to about 250° F. A wax matrix containing the carboxylic acid losartan is added as the temperature of the mix is lowered to about 120° F. to about 194° F., whereupon additional ingredients such as flavors, colorants, and preservatives are added. The formulation is further cooled and formed to pieces of desired dimensions.

In another embodiment, an oral dosage form comprises a non-chewable, fast dissolving dosage form of the carboxylic acid losartan. These dosage forms are made by methods known to those of ordinary skill in the art of pharmaceutical formulations. For example, Cima Labs has produced oral dosage forms including microparticles and effervescents that rapidly disintegrate in the mouth and provide adequate taste-masking. Zydis (ZYPREXA®) is produced by Eli Lilly as in a rapidly dissolvable, freeze-dried, sugar matrix formulated as a rapidly dissolving tablet. U.S. Pat. No. 5,178,878 and U.S. Pat. No. 6,221,392 provide teachings regarding fast-dissolve dosage forms, and are incorporated by reference.

An exemplary fast dissolve dosage form includes a mixture incorporating a water and/or saliva activated effervescent disintegration agent and microparticles. The microparticles incorporate carboxylic acid losartan together with a protective material substantially encompassing the carboxylic acid losartan. The term “substantially encompassing” as used in this context means that the protective material substantially shields the carboxylic acid losartan from contact with the environment outside of the microparticle. Thus, each microparticle incorporates a discrete mass of the carboxylic acid losartan covered by a coating of the protective material, in which case the microparticle can be referred to as a “microcapsule”. Alternatively or additionally, each microparticle has the carboxylic acid losartan dispersed or dissolved in a matrix of the protective material. The mixture including the microparticles and effervescent agent is present as a tablet of a size and shape adapted for direct oral administration to a patient, such as a human patient. The tablet is substantially completely disintegrable upon exposure to water and/or saliva. The effervescent disintegration agent is present in an amount effective to aid in disintegration of the tablet, and to provide a distinct sensation of effervescence when the tablet is placed in the mouth of a patient.

The effervescent sensation is not only pleasant to the patient but also tends to stimulate saliva production, thereby providing additional water to aid in further effervescent action. Thus, once the tablet is placed in the patient's mouth, it will disintegrate rapidly and substantially completely without any voluntary action by the patient. Even if the patient does not chew the tablet, disintegration will proceed rapidly. Upon disintegration of the tablet, the microparticles are released and can be swallowed as a slurry or suspension of the microparticles. The microparticles thus may be transferred to the patient's stomach for dissolution in the digestive tract and systemic distribution of the pharmaceutical ingredient.

The term effervescent disintegration agent(s) includes compounds which evolve gas. Suitable effervescent agents evolve gas by means of chemical reactions which take place upon exposure of the effervescent disintegration agent to water and/or to saliva in the mouth. The bubble or gas generating reaction is most often the result of the reaction of a soluble acid source and an alkali metal carbonate or carbonate source. The reaction of these two general classes of compounds produces carbon dioxide gas upon contact with water included in saliva.

Such water activated materials should be kept in a generally anhydrous state with little or no absorbed moisture or in a stable hydrated form since exposure to water will prematurely disintegrate the tablet. The acid sources or acid are those which are safe for human consumption and generally include food acids, acid anhydrides and acid salts. Food acids include citric acid, tartaric acid, malic acid, fumaric acid, adipic acid, succinic acid, and combinations comprising one or more of the foregoing acids. Because these acids are directly ingested, their overall solubility in water is less important than it would be if the effervescent tablet formulations were intended to be dissolved in a glass of water. Acid anhydrides of the above described acids may also be used. Acid salts include sodium, dihydrogen phosphate, disodium dihydrogen pyrophosphate, acid citrate salts, sodium acid sulfite, and combinations comprising one or more of the foregoing acid salts.

Carbonate sources include dry solid carbonate and bicarbonate salts such as sodium bicarbonate, sodium carbonate, potassium bicarbonate and potassium carbonate, magnesium carbonate and sodium sesquicarbonate, sodium glycine carbonate, L-lysine carbonate, arginine carbonate, amorphous calcium carbonate, and combinations comprising one or more of the foregoing carbonates.

The effervescent disintegration agent is not always based upon a reaction which forms carbon dioxide. Reactants which evolve oxygen or other gasses which are pediatrically safe may also be employed. Where the effervescent agent includes two mutually reactive components, such as an acid source and a carbonate source, it is preferred that both components react substantially completely. Therefore, an equivalent ratio of components which provides for equal equivalents is preferred. For example, if the acid used is diprotic, then either twice the amount of a mono-reactive carbonate base, or an equal amount of a di-reactive base should be used for complete neutralization to be realized. However, the amount of either acid or carbonate source may exceed the amount of the other component. This may be useful to enhance taste and/or performance of a tablet containing an overage of either component. In this case, it is acceptable that the additional amount of either component may remain unreacted.

In general, the amount of effervescent disintegration agent useful for the formation of tablets is about 5 to about 50 wt % of the final composition, specifically about 15 and about 30 wt %, and most specifically about 20 and about 25 wt %.

More specifically, the tablets should contain an amount of effervescent disintegration agent effective to aid in the rapid and complete disintegration of the tablet when orally administered. By “rapid”, it is understood that the tablets should disintegrate in the mouth of a patient in less than about 10 minutes, and desirably between about 30 seconds and about 7 minutes, preferably tablet should dissolve in the mouth in between about 30 seconds and about 5 minutes. Disintegration time in the mouth can be measured by observing the disintegration time of the tablet in water at about 37° C. The tablet is immersed in the water without forcible agitation. The disintegration time is the time from immersion for substantially complete dispersion of the tablet as determined by visual observation. As used herein, the term “complete disintegration” of the tablet does not require dissolution or disintegration of the microcapsules or other discrete inclusions.

The carboxylic acid losartan in the dosage form is optionally present in microparticles. Each microparticle incorporates the carboxylic acid losartan in conjunction with a protective material. The microparticle may be provided as a microcapsule or as a matrix-type microparticle. Microcapsules may incorporate a discrete mass of the carboxylic acid losartan surrounded by a discrete, separately observable coating of the protective material. Conversely, in a matrix-type particle, the carboxylic acid losartan is dissolved, suspended or otherwise dispersed throughout the protective material. Certain microparticles include attributes of both microcapsules and matrix-type particle. For example, a microparticle may incorporate a core incorporating a dispersion of the carboxylic acid losartan in a first protective material and a coating of a second protective material, which is the same as or different from the first protective material surrounding the core. Alternatively, a microparticle incorporates a core consisting essentially of the carboxylic acid losartan and a coating incorporating the protective material, the coating itself having some of the pharmaceutical ingredient dispersed within it.

The microparticles are about 75 to 600 microns mean outside diameter, and more preferably about 150 to about 500 microns. Microparticles above about 200 microns may be employed. Thus, the microparticles are about 200 mesh to about 30 mesh U.S. standard size, and more specifically about 100 mesh to about 35 mesh.

Tablets can be manufactured by well-known tableting procedures. In common tableting processes, the material which is to be tableted is deposited into a cavity, and one or more punch members are then advanced into the cavity and brought into intimate contact with the material to be pressed, whereupon compressive force is applied. The material is thus forced into conformity with the shape of the punches and the cavity. Hundreds, and even thousands, of tablets per minute can be produced in this fashion.

Another exemplary fast-dissolve dosage form is a hard, compressed, rapidly dissolvable dosage form adapted for direct oral dosing. The dosage form includes carboxylic acid losartan often in the form of a protected particle, and a matrix. The matrix includes a nondirect compression filler and a lubricant, although, it may include other ingredients as well. The dosage form is adapted to rapidly dissolve in the mouth of a patient, yet it has a friability of about 2% or less when tested according to the U.S.P. Generally, the dosage form will also have a hardness of at least about 15-2 Newtons (1.5-2.0 kilopond (kp)). Not only does the dosage form dissolve quickly, it does so in a way that provides a positive organoleptic sensation to the patient. In particular, the dosage form dissolves with a minimum of unpleasant grit which is tactilely inconsistent with a positive organoleptic sensation to the patient.

Suitable protective materials include polymers utilized in the formation of microparticles, matrix-type microparticles and microcapsules. Among these polymers are cellulosic materials such as naturally occurring cellulose and synthetic cellulose derivatives; acrylic polymers; and vinyl polymers. Other suitable polymers include proteinaceous materials such as gelatin, polypeptides and natural and synthetic shellacs and waxes. Protective polymers also include ethylcellulose, methylcellulose, carboxymethyl cellulose and acrylic resin material sold under the registered trademark EUDRAGIT® by Rohm Pharma GmbH of Darmstadt, Germany.

Generally, when a coating is used, the coating comprises greater than or equal to about 5 wt % based on the weight of the resulting particles. More specifically, the coating constitutes at least about 10 wt % by weight of the particle. The upper limit of protective coating material used is generally less critical, except that where a rapid release of the active ingredient is desired, the amount of coating material should not be so great that the coating material impedes the release profile of the carboxylic acid losartan when ingested. Thus, it may be possible to use greater than 100 percent of the weight of the core, thereby providing a relatively thick coating.

Suitable fillers include nondirect compression fillers. Exemplary fillers include, for example, nondirect compression sugars and sugar alcohols. Such sugars and sugar alcohols include, without limitation, dextrose, mannitol, sorbitol, lactose and sucrose. Of course, dextrose, for example, can exist as either a direct compression sugar, i.e., a sugar which has been modified to increase its compressibility, or a nondirect compression sugar.

Generally, the balance of the formulation is the matrix. Thus the percentage of filler can approach 100% by weight. However, generally, the amount of nondirect compression filler is about 25 to about 95 wt %, specifically about 50 to about 95 wt % and more specifically about 60 to about 95 wt % of the total weight of the dosage form.

In the fast-dissolve dosage form, a relatively high proportion of lubricant may be employed. Lubricants, and in particular, hydrophobic lubricants such as magnesium stearate, are generally used in an amount of about 0.25 to about 5 wt %, according to the Handbook of Pharmaceutical Excipients. Specifically, the amount of lubricant used can be about 1 to about 2.5 wt %, and more preferably about 1.5 to about 2 wt %. Despite the use of this relatively high rate of lubricant, the formulations exhibit a superior compressibility, hardness, and rapid dissolution within the mouth.

Hydrophobic lubricants include, for example, alkaline stearates, stearic acid, mineral and vegetable oils, glyceryl behenate, sodium stearyl fumarate, and combinations comprising one or more of the foregoing lubricants. Hydrophilic lubricants can also be used.

The hard, compressed dosage forms have a hardness of at least about 15 Newtons and are designed to dissolve spontaneously and rapidly in the mouth of a patient in less than about 90 seconds to thereby liberate the particles. Preferably the dosage form will dissolve in less than about 60 seconds and even more preferably about 45 seconds. This measure of hardness is based on the use of small tablets of less than about 0.25 inches in diameter. A hardness of at least about 20 Newtons is preferred for larger tablets. Direct compression techniques are preferred for the formation of the tablets.

In one embodiment, the carboxylic acid losartan dosage form comprises a taste-masked dosage form. The taste-masked dosage forms may be liquid dosage forms such as those disclosed in U.S. Pat. No.6,197,348, incorporated herein by reference.

In one embodiment, a solid taste masked dosage form comprises a core element comprising the carboxylic acid losartan and a coating surrounding the core element. The core element comprising the carboxylic acid losartan is in the form of a capsule or is encapsulated by micro-encapsulation techniques, where a polymeric coating is applied to the formulation. The core element includes the carboxylic acid losartan and optionally also includes pharmaceutically acceptable carriers or excipients, fillers, flavoring agents, stabilizing agents and/or colorants.

The taste masked dosage form may include about 77 wt % to about 100 wt %, specifically about 80 wt % to about 90 wt %, based on the total weight of the composition of the core element including the carboxylic acid losartan; and about 20 wt % to about 70 wt %, of a substantially continuous coating on the core element formed from a coating material including a polymer. The core element includes about 52 wt % to about 85 wt % of the carboxylic acid losartan; and about 5 wt % to about 25 wt % of a supplementary component selected from waxes, water insoluble polymers, enteric polymers, and partially water soluble polymers, other suitable pharmaceutical excipients, and combinations comprising one or more of the foregoing components.

The core element optionally includes pharmaceutically acceptable carriers or excipients, fillers, flavoring agents, stabilizing agents, colorants, and combinations comprising one or more of the foregoing additives. Suitable fillers include, for example, insoluble materials such as silicon dioxide, titanium dioxide, talc, alumina, starch, kaolin, polacrilin potassium, powdered cellulose, and microcrystalline cellulose, and combinations comprising one or more of the foregoing fillers. Soluble fillers include, for example, mannitol, sucrose, lactose, dextrose, sodium chloride, sorbitol, and combinations comprising one or more of the foregoing fillers. The filler may be present in amounts of up to about 75 wt % based on the total weight of the composition. The particles of the core element may be in the range of the particle size set forth above for core particles of core elements.

The core element is optionally in the form of a powder, for example, having particle sizes of about 35 μm to about 125 μm. The small particle size facilitates a substantially non-gritty feel in the mouth. Small particle size also minimizes break-up of the particles in the mouth, e.g., by the teeth. When in the form of a powder, the taste masked dosage form may be administered directly into the mouth or mixed with a carrier such as water, or semi-liquid compositions such as syrups, yogurt, and the like. However, the taste masked carboxylic acid losartan may be provided in any suitable unit dosage form.

The coating material of the taste-masked formulation may take a form that provides a substantially continuous coating and still provides taste masking. In some cases, the coating also provides controlled-release of the carboxylic acid losartan. The polymer used in taste masked dosage form coating may be a water insoluble polymer such as, for example, ethyl cellulose. The coating material of the taste masked dosage form may further include a plasticizer.

A method of preparing taste-masked pharmaceutical formulations such as powdered formulations includes mixing a core element and a coating material in a diluent and spray drying the mixture to form a taste-masked formulation. Spray drying of the carboxylic acid losartan and polymer in the solvent involves spraying a stream of air into an atomized suspension so that solvent is caused to evaporate leaving the carboxylic acid losartan coated with the polymer coating material.

For a solvent such as methylene chloride, the solvent concentration in the drying chamber is typically maintained above about 40,000 parts, or about 40,000 to about 100,000 parts per million of organic solvent. The spray-drying process for such solvents is conducted at a process temperature of about 5° C. to about 35° C. Spray drying of the dosage forms is undertaken, for example, utilizing either rotary, pneumatic or pressure atomizers located in either a co-current, counter-current or mixed-flow spray dryer or variations thereof. The drying gas is optionally heated or cooled to control the rate of drying. A temperature below the boiling point of the solvent may be used. Inlet temperatures are about 40° C. to about 120° C. and outlet temperatures about 5° C. to about 35° C.

The coat formation may be optimized to meet the needs of the material or application. Controlling the process parameters including temperature, solvent concentration, spray dryer capacity, atomizing air pressure, droplet size, viscosity, total air pressure in the system and solvent system, allows the formation of a range of coats, ranging from dense, continuous, non-porous coats through to more porous microcapsule/polymer matrices.

An optional post-treatment step is used to remove residual solvent. The post treatment may include a post drying step including drying the final product on a tray and drying the product at a bed temperature sufficient to remove excess solvent, but not degrade the carboxylic acid losartan. Preferably the drying temperature is about 35° C. to about 4° C. Once completed, the product may be collected by a suitable method, such as collection by sock filters or cyclone collection.

In one embodiment, liquid dosage forms of the carboxylic acid losartan may be formulated that also provide adequate taste masking. A taste masked liquid dosage form comprises, for example, a suspension of microcapsules taste masked as a function of the pH of a suspending medium and a polymer coating. Many active agents are less soluble at higher or lower pH than at the pH value of the mouth, which is around 5.9. In these cases, the active agent is insufficiently solubilized to be tasted if the equilibrium concentration is below the taste threshold. However, problems can arise if all of the suspended particles are not swallowed because the active agent which remains in the mouth is able to dissolve at the pH of the mouth. The use of polymeric coatings on the active agent particles, which inhibit or retard the rate of dissolution and solubilization of the active agent is one means of overcoming the taste problems with delivery of active agents in suspension. The polymeric coating allows time for all of the particles to be swallowed before the taste threshold concentration is reached in the mouth.

Optimal taste masked liquid formulations are obtained when consideration is given to: (i) the pH of maximum insolubility of the active agent; (ii) the threshold concentration for taste of the active agent; (iii) the minimum buffer strength required in the medium to avoid delayed or after taste; (iv) the pH limit beyond which further increase or decrease of pH leads to unacceptable instability of the active agent; and (v) the compatibility and chemical, physical and microbial stability of the other ingredients to the pH values of the medium.

In one embodiment, a taste masked liquid dosage form comprises the carboxylic acid losartan, a polymer with a quaternary ammonium functionality encapsulating the carboxylic acid losartan, and a suspending medium adjusted to a pH at which the carboxylic acid losartan remains substantially insoluble, for suspending the encapsulated carboxylic acid losartan. The carboxylic acid losartan is taste masked by the combination of the polymer and suspending medium.

The carboxylic acid losartan may be in the form of its neutral or salt form and is further in the form of particles, crystals, microcapsules, granules, microgranules, powders, pellets, amorphous solids or precipitates. The particles optionally further include other functional components. The carboxylic acid losartan may have a defined particle size distribution, specifically about 0.1 to about 500 μm, more specifically about 1 to about 250 μm, and most specifically about 10 to about 150 μm, where there is acceptable mouth feel and little chance of chewing on the residual particles and releasing the carboxylic acid losartan to taste.

The taste masked liquid dosage form optionally includes, along with the carboxylic acid losartan, other functional components present for the purpose of modifying the physical, chemical, or taste properties of the carboxylic acid losartan. For example, the carboxylic acid losartan may be in the form of ion-exchange or cyclodextrin complexes or the carboxylic acid losartan may be included as a mixture or dispersion with various additives such as waxes, lipids, dissolution inhibitors, taste-masking or -suppressing agents, pharmaceutically acceptable carriers or excipients, fillers, and combinations comprising one or more of the foregoing components.

In one embodiment, the polymer used to encapsulate the carboxylic acid losartan or the pharmaceutical unit is a polymer having a quaternary ammonium functionality, i.e., a polymer having quaternary ammonium groups on the polymer backbone. These polymers are effective in preventing the taste perception of the carboxylic acid losartan when the resulting microcapsules are formulated as suspensions and stored for long periods despite their widely recognized properties of being permeable to water and dissolved carboxylic acid losartan. A suitable polymer is a copolymer of acrylic and methacrylic acid esters with quaternary ammonium groups. The polymer may be a copolymer of methyl methacrylate and triethylammonium methacrylate. Specific examples of suitable polymers include EUDRAGIT® RS and EUDRAGIT® RL, available from Rohm America, LLC, Piscataway, N.J., used individually or in combination to change the permeability of the coat. A polymer coat having a blend of the RS or RL polymer along with other pharmaceutically acceptable polymers may also be employed. The other polymers may be cellulose ethers such as ethyl cellulose, cellulose esters such as cellulose acetate and cellulose propionate, polymers that dissolve at acidic or alkaline pH, such as EUDRAGIT® E, cellulose acetate phthalate, hydroxypropylmethyl cellulose phthalate, and combinations comprising one or more of the foregoing polymers.

The quantity of polymer used in relation to the carboxylic acid losartan is about 0.01-10:1, preferably about 0.02-1:1, more preferably about 0.03-0.5:1 and most preferably about 0.05-0.3:1 by weight.

The carboxylic acid losartan particles are suspended, dispersed or emulsified in the suspending medium after encapsulation with the polymer. Suitable suspending media include water-based media, but may be a non-aqueous carrier as well, constituted at an optimum pH for the carboxylic acid losartan or pharmaceutical unit, such that the carboxylic acid losartan remains substantially insoluble. The pH and ionic strength of the medium are selected on the basis of stability, solubility and taste threshold to provide the optimum taste masking effect, and which is compatible with the stability of the carboxylic acid losartan the polymer coat and the coating excipients.

Buffering agents are optionally included in the suspending medium for maintaining the desired pH. Suitable buffering agents include dihydrogen phosphate, hydrogen phosphate, amino acids, citrate, acetate, phthalate, tartrate salts of the alkali or alkaline earth metal cations such as sodium, potassium, magnesium, calcium, and combinations comprising one or more of the foregoing buffering agents. The buffering agents are used in a suitable combination for achieving the required pH and are typically of a buffer strength of about 0.01 to about 1 moles/liter of the final formulation, specifically about 0.01 to about 0.1 moles/liter, and most specifically about 0.02 to about 0.05 moles/liter.

The taste masked liquid dosage form optionally further includes other optional dissolved or suspended agents to provide stability to the suspension. These agents include suspending agents or stabilizers such as, for example, methyl cellulose, sodium alginate, xanthan gum, (poly)vinyl alcohol, microcrystalline cellulose, colloidal silicas, bentonite clay, and combinations comprising one or more of the foregoing agents. Other agents used include preservatives such as methyl, ethyl, propyl and butyl parabens, sweeteners such as saccharin sodium, aspartame, mannitol, flavorings such as grape, cherry, peppermint, menthol and vanilla flavors, and antioxidants or other stabilizers, and combinations comprising one or more of the foregoing agents.

A method of preparing a taste masked dosage form for oral delivery, comprises encapsulating the carboxylic acid losartan with a polymer having a quaternary ammonium functionality; and adding a suspending medium adjusted to a pH at which the carboxylic acid losartan is substantially insoluble, for suspending the encapsulated carboxylic acid losartan; wherein the carboxylic acid losartan is taste masked by the combination of the polymer and the medium. In the process, the polymer for encapsulation of the carboxylic acid losartan or carboxylic acid losartan -containing particle is dissolved in a solution or solvent chosen for its poor solubility for the carboxylic acid losartan and good solubility for the polymer. Examples of appropriate solvents include but are not limited to methanol, ethanol, isopropanol, chloroform, methylene chloride, cyclohexane, and toluene, either used in combination or used alone. Aqueous dispersions of polymers may also be used for forming the carboxylic acid losartan microparticles.

Encapsulation of the carboxylic acid losartan or pharmaceutical unit by the polymer may be performed by a method such as suspending, dissolving, or dispersing the carboxylic acid losartan in a solution or dispersion of polymer coating material and spray drying, fluid-bed coating, simple or complex coacervation, coevaporation, co-grinding, melt dispersion and emulsion-solvent evaporation techniques, and the like.

The polymer coated carboxylic acid losartan powder can also be employed as an alternative be applied for the preparation of reconstitutable powders, i.e.; dry powder carboxylic acid losartan products that are reconstituted as suspensions in a liquid vehicle such as water before usage. The reconstitutable powders have a long shelf life and the suspensions, once reconstituted, have adequate taste masking.

In one embodiment, the carboxylic acid losartan dosage form is a sprinkle dosage form. Sprinkle dosage forms include particulate or pelletized forms of the carboxylic acid losartan, optionally having functional or non-functional coatings, with which a patient or a caregiver can sprinkle the particulate/pelletized dose into drink or onto soft food. A sprinkle dosage form comprises particles of about 10 to about 100 micrometers in their major dimension. Sprinkle dosage forms are in the form of optionally coated granules or as microcapsules. Sprinkle dosage forms may be immediate or controlled-release formulations such as sustained-release formulations. See U.S. Pat. No. 5,084,278, which is hereby incorporated by reference for its teachings regarding microcapsule formulations, which may be administered as sprinkle dosage forms.

In one embodiment, a carboxylic acid losartan dosage form is suitable for buccal or sublingual delivery. For delivery to the buccal or sublingual membranes, an oral formulation, such as a lozenge, tablet, or capsule, is employed. The method of manufacture of these formulations is known in the art, including, but not limited to, the addition of the carboxylic acid losartan to a pre-manufactured tablet; cold compression of an inert filler, a binder, and either a pharmacological agent or a substance containing the agent (as described, for example, in U.S. Pat. No. 4,806,356, incorporated herein by reference); and encapsulation. Another oral formulation is one that can be applied with an adhesive, such as the cellulose derivative hydroxypropyl cellulose, to the oral mucosa, for example as described in U.S. Pat. No. 4,940, 587, incorporated herein by reference. This buccal adhesive formulation, when applied to the buccal mucosa, allows for controlled release of the pharmacological agent into the mouth and through the buccal mucosa.

In another embodiment, a carboxylic acid losartan dosage form comprises a zero order release dosage form. In zero order release, the amount of drug release remains constant with respect to time. Suitable methods for preparing zero order controlled release dosage forms include those operating by a rate-controlling membrane and by osmotic pumps, and wax matrix dosage forms, optionally comprising a coating.

In another embodiment, a carboxylic acid losartan dosage form is an “osmotic pump” dosage form such as one formulated with OROS® technology (Alza Corporation, Mountain View, Calif.). Such dosage forms have a fluid-permeable (semipermeable) membrane wall, an osmotically active expandable driving member (the osmotic push layer), and a density element for delivering the carboxylic acid losartan. In an osmotic pump dosage form, the active material is dispensed through an exit means comprising a passageway, orifice, or the like, by the action of the osmotically active driving member. The carboxylic acid losartan of the osmotic pump dosage form is, for example, formulated as a thermo-responsive formulation in which the carboxylic acid losartan is dispersed in a thermo-responsive composition. Alternatively, the osmotic pump dosage form contains a thermo-responsive element comprising a thermo-responsive composition at the interface of the osmotic push layer and the carboxylic acid losartan composition.

The osmotic pump dosage form comprises a semipermeable membrane. The capsule or other dispenser of the osmotic pump dosage form can be provided with an outer wall comprising the selectively semipermeable material. A selectively permeable material is one that does not adversely affect a host or animal, is permeable to the passage of an external aqueous fluid, such as water or biological fluids, while remaining essentially impermeable to the passage of the carboxylic acid losartan, and maintains its integrity in the presence of a thermotropic thermo-responsive composition, that is it does not melt or erode in its presence. The selectively semipermeable material forming the outer wall is substantially insoluble in body fluids, nontoxic, and non-erodible.

Representative materials for forming the selectively semipermeable wall include semipermeable homopolymers, semipermeable copolymers, and the like. Suitable materials include, for example, cellulose esters, cellulose monoesters, cellulose diesters, cellulose triesters, cellulose ethers, cellulose ester-ethers, and combinations comprising one or more of the foregoing materials. These cellulosic polymers have a degree of substitution, D.S., on their anhydroglucose unit from greater than 0 up to 3 inclusive. By degree of substitution is meant the average number of hydroxyl groups originally present on the anhydroglucose unit that are replaced by a substituting group, or converted into another group. The anhydroglucose unit can be partially or completely substituted with groups such as acyl, alkanoyl, aroyl, alkyl, alkenyl, alkoxy, halogen, carboalkyl, alkylcarbamate, alkylcarbonate, alkylsulfonate, alkylsulfamate, and like semipermeable polymer forming groups.

Other selectively semipermeable materials include, for example, cellulose acylate, cellulose diacylate, cellulose triacylate, cellulose acetate, cellulose diacetate, cellulose triacetate, mono-, di- and tri-cellulose alkanylates, mono-, di- and tri-alkenylates, mono-, di- and tri-aroylates, and the like, and combinations comprising one or more of the foregoing materials. Exemplary polymers including cellulose acetate having a D.S. of 1.8 to 2.3 and an acetyl content of about 32 to about 39.9%; cellulose diacetate having a D.S. of 1 to 2 and an acetyl content of about 21 to about 35%; cellulose triacetate having a D.S of 2 to 3 and an acetyl content of about 34 to about 44.8%, and the like. More specific cellulosic polymers include cellulose propionate having a D.S. of 1.8 and a propionyl content of about 38.5%; cellulose acetate propionate having an acetyl content of about 1.5 to about 7% and an propionyl content of about 39 to about 42%; cellulose acetate propionate having an acetyl content of about 2.5 to about 3%, an average propionyl content of about 39.2 to about 45% and a hydroxyl content of about 2.8 to about 5.4%; cellulose acetate butyrate having a D.S. of 1.8, an acetyl content of about 13 to about 15%, and a butyryl content of about 34 to about 39%; cellulose acetate butyrate having an acetyl content of about 2 to about 29.5%, a butyryl content of about 17 to about 53%, and a hydroxyl content of about 0.5 to about 4.7%; cellulose triacylates having a D.S. of 2.9 to 3 such as cellulose trivalerate, cellulose trilaurate, cellulose tripalmitate, cellulose trioctanoate, and cellulose tripropionate; cellulose diesters having a D.S. of 2.2 to 2.6 such as cellulose disuccinate, cellulose dipalmitate, cellulose dioctanoate, cellulose dicarpylate and the like; mixed cellulose esters such as cellulose acetate valerate, cellulose acetate succinate, cellulose propionate succinate, cellulose acetate octanoate, cellulose valerate palmitate, cellulose acetate heptonate, and the like, and combinations comprising one or more of the foregoing polymers.

Additional selectively semipermeable polymers include, for example, acetaldehyde dimethyl cellulose acetate, cellulose acetate ethylcarbamate, cellulose acetate methylcarbamate, cellulose dimethylaminoacetate, semi-permeable polyamides, semipermeable polyurethanes, semi-permeable polysulfanes, semipermeable sulfonated polystyrenes, cross-linked, selectively semipermeable polymers formed by the coprecipitation of a polyanion and a polycation, selectively semipermeable silicon rubbers, semipermeable polystyrene derivates, semipermeable poly(sodium styrenesulfonate), semipermeable poly(vinylbenzyltrimethyl) ammonium chloride polymers, and combinations comprising one or more of the foregoing polymers.

The osmotically expandable driving member, or osmotic push layer, of the soft capsule osmotic pump dosage form is a swellable and expandable inner layer. The materials used for forming the osmotic push layer are neat polymeric materials and/or polymeric materials blended with osmotic agents that interact with water or a biological fluid, absorb the fluid, and swell or expand to an equilibrium state. The polymer should exhibit the ability to retain a significant fraction of imbibed fluid in the polymer molecular structure. Such polymers may be, for example, gel polymers that can swell or expand to a very high degree, usually exhibiting about a 2 to 50-fold volume increase. Swellable, hydrophilic polymers, also known as osmopolymers, can be non-cross-linked or lightly cross-linked. The cross-links can be covalent or ionic bonds with the polymer possessing the ability to swell but not dissolve in the presence of fluid. The polymer can be of plant, animal or synthetic origin. Polymeric materials useful for the present purpose include poly(hydroxyalkyl methacrylate) having a molecular weight of about 5,000 to about 5,000,000, poly(vinylpyrrolidone) having a molecular weight of about 10,000 to about 360,000, anionic and cationic hydrogels, poly(electrolyte) complexes, poly(vinyl alcohol) having a low acetate residual, a swellable mixture of agar and carboxymethyl cellulose, a swellable composition comprising methyl cellulose mixed with a sparingly crosslinked agar, a water-swellable copolymer produced by a dispersion of finely divided copolymer of maleic anhydride with styrene, ethylene, propylene, or isobutylene, water swellable polymer of N-vinyl lactams, and the like, and combinations comprising one or more of the foregoing polymers. Other gelable, fluid imbibing and retaining polymers useful for forming the osmotic push layer include pectin having a molecular weight ranging of about 30,000 to about 300,000, polysaccharides such as agar, acacia, karaya, tragacanth, algins and guar, acidic carboxy polymer and its salt derivatives, polyacrylamides, water-swellable indene maleic anhydride polymers; polyacrylic acid having a molecular weight of about 80,000 to about 200,000; POLYOX™, polyethylene oxide polymers having a molecular weight of about 100,000 to about 5,000,000, and greater, starch graft copolymers, polyanions and polycations exchange polymers, starch-polyacrylonitrile copolymers, acrylate polymers with water absorbability of about 400 times its original weight, diesters of polyglucan, a mixture of cross-linked polyvinyl alcohol and poly(N-vinyl-2-pyrrolidone), zein available as prolamine, poly(ethylene glycol) having a molecular weight of about 4,000 to about 100,000, and the like, and combinations comprising one or more of the foregoing polymers.

The osmotically expandable driving layer of the osmotic pump dosage form may further contain an osmotically effective compound (osmagent) that can be used neat or blended homogeneously or heterogeneously with the swellable polymer, to form the osmotically expandable driving layer. Such osmagents include osmotically effective solutes that are soluble in fluid imbibed into the swellable polymer, and exhibit an osmotic pressure gradient across the semipermeable wall against an exterior fluid. Suitable osmagents include, for example, solid compounds such as magnesium sulfate, magnesium chloride, sodium chloride, lithium chloride, potassium sulfate, sodium sulfate, mannitol, urea, sorbitol, inositol, and the like, and combinations comprising one or more of the foregoing osmagents. The osmotic pressure in atmospheres, atm, of the osmagents may be greater than about zero atm, and generally about zero atm to about 500 atm, or higher.

The swellable, expandable polymer of the osmotically expandable driving layer, in addition to providing a driving source for delivering the carboxylic acid losartan from the dosage form, may also function as a supporting matrix for an osmotically effective compound. The osmotic compound can be homogeneously or heterogeneously blended with the polymer to yield the desired expandable wall or expandable pocket. The composition in a presently preferred embodiment comprises (a) at least one polymer and at least one osmotic compound, or (b) at least one solid osmotic compound. Generally, a composition comprises about 20% to about 90% by weight of polymer and about 80% to about 10% by weight of osmotic compound, specifically about 35% to about 75% by weight of polymer and about 65% to about 25% by weight of osmotic compound.

The carboxylic acid losartan of the osmotic pump dosage form may be formulated as a thermo-responsive formulation in which the carboxylic acid losartan is dispersed in a thermo-responsive composition. Alternatively, the osmotic pump dosage form may contain a thermo-responsive element comprising a thermo-responsive composition at the interface of the osmotic push layer and the carboxylic acid losartan composition. Representative thermo-responsive compositions and their melting points are as follows: Cocoa butter (32° C.-34° C.), cocoa butter plus 2% beeswax (35° C.-37° C.), propylene glycol monostearate and distearate (32° C.-35° C.), hydrogenated oils such as hydrogenated vegetable oil (36° C.-37.5° C.), 80% hydrogenated vegetable oil and 20% sorbitan monopalmitate (39° C.-39.5° C.), 80% hydrogenated vegetable oil and 20% polysorbate 60, (36° C.-37° ), 77.5% hydrogenated vegetable oil, 20% sorbitan trioleate, 2.5% beeswax and 5.0% distilled water, (37° C.-38° C.), mono-, di-, and triglycerides of acids having from 8-22 carbon atoms including saturated and unsaturated acids such as palmitic, stearic, oleic, lineolic, linolenic and archidonic; triglycerides of saturated fatty acids with mono- and diglycerides (34° C.-35.5° C.), propylene glycol mono- and distearates 3(33° C.-34° C.), partially hydrogenated cottonseed oil (35° C.-39° C.), a block polymer of polyoxy-alkylene and propylene glycol; block polymers comprising 1,2-butylene oxide to which is added ethylene oxide; block copolymers of propylene oxide and ethylene oxide, hardened fatty alcohols and fats (33° C.-36° C.), hexadienol and hydrous lanolin triethanolamine glyceryl monostearate (38° C.), eutectic mixtures of mono-, di-, and triglycerides (35° C.-39° C.), WITEPSOL#15, triglyceride of saturated vegetable fatty acid with monoglycerides (33.5° C.-35.5° C.), WITEPSOL H32 free of hydroxyl groups (31° C.-33° C.), WITEPSOL W25 having a saponification value of 225-240 and a melting point of (33.5° C.-35.5° C.), WITEPSOL E75 having a saponification value of 220-230 and a melting point of (37° C.-39° C.), a polyalkylene glycol such as polyethylene glycol 1000, a linear polymer of ethylene oxide (38° C.-41° C.), polyethylene glycol 1500 (38° C.-41° C.), polyethylene glycol monostearate (39° C.-42.5° C.), 33% polyethylene glycol 1500, 47% polyethylene glycol 6000 and 20% distilled water (39° C.-41° C.), 30% polyethylene glycol 1500, 40% polyethylene glycol 4000 and 30% polyethylene glycol 400, (33° C.-38° C.), mixture of mono-, di-, and triglycerides of saturated fatty acids having 11 to 17 carbon atoms, (33° C.-35° C.), and the like. The thermo-responsive compositions, including thermo-responsive carriers are useful for storing the carboxylic acid losartan in a solid composition at a temperature of about 20° C. to about 33° C., maintaining an immiscible boundary at the swelling composition interface, and for dispensing the agent in a flowable composition at a temperature greater than about 33° C. and preferably between about 33° C. and about 40° C.

The amount of carboxylic acid losartan present in the osmotic pump dosage form is about 20 mg to about 150 mg or more. The osmotic dosage form may be formulated for once daily or less frequent administration.

The carboxylic acid losartan of the osmotic pump dosage form is formulated by a number of techniques known in the art for formulating solid and liquid oral dosage forms. The carboxylic acid losartan of the osmotic pump dosage form may be formulated by wet granulation. In an exemplary wet granulation method, the carboxylic acid losartan and the ingredients comprising the carboxylic acid losartan layer are blended using an organic solvent, such as isopropyl alcohol-ethylene dichloride 80:20 v:v (volume:volume) as the granulation fluid. Other granulating fluids such as denatured alcohol 100% may be used for this purpose. The ingredients forming the carboxylic acid losartan layer are individually passed through a screen such as a 40-mesh screen and then thoroughly blended in a mixer. Next, other ingredients comprising the carboxylic acid losartan layer are dissolved in a portion of the granulation fluid, such as the cosolvent described above. Then the latter prepared wet blend is slowly added to the carboxylic acid losartan blend with continual mixing in the blender. The granulating fluid is added until a wet blend is produced, which wet mass then is forced through a screen such as a 20-mesh screen onto oven trays. The blend is dried for about 18 to about 24 hours at about 30° C. to about 50° C. The dry granules are sized then with a screen such as a 20-mesh screen. Next, a lubricant is passed through a screen such as an 80-mesh screen and added to the dry screen granule blend. The granulation is put into milling jars and mixed on a jar mill for about 1 to about 15 minutes. The push layer may also be made by the same wet granulation techniques. The compositions are pressed into their individual layers in a KILIAN press-layer press.

Another manufacturing process that can be used for providing the carboxylic acid losartan layer and osmotically expandable driving layer comprises blending the powered ingredients for each layer independently in a fluid bed granulator. After the powered ingredients are dry blended in the granulator, a granulating fluid, for example, poly(vinyl-pyrrolidone) in water, or in denatured alcohol, or in 95:5 ethyl alcohol/water, or in blends of ethanol and water is sprayed onto the powders. Optionally, the ingredients are dissolved or suspended in the granulating fluid. The coated powders are then dried in a granulator. This process granulates the ingredients present therein while adding the granulating fluid. After the granules are dried, a lubricant such as stearic acid or magnesium stearate is added to the granulator. The granules for each separate layer are pressed then in the manner described above.

The carboxylic acid losartan formulation and osmotic push layer of the osmotic dosage form may also be manufactured by mixing carboxylic acid losartan with composition forming ingredients and pressing the composition into a solid lamina possessing dimensions that correspond to the internal dimensions of the compartment. In another manufacture, the carboxylic acid losartan and other carboxylic acid losartan composition-forming ingredients and a solvent are mixed into a solid, or a semisolid, by methods such as ballmilling, calendaring, stirring or rollmilling, and then pressed into a preselected layer forming shape. Next, a layer of a composition comprising an osmopolymer and an optional osmagent are placed in contact with the layer comprising the carboxylic acid losartan. The layering of the first layer comprising the carboxylic acid losartan and the second layer comprising the osmopolymer and optional osmagent composition can be accomplished by using a conventional layer press technique. The semipermeable wall can be applied by molding, spraying or dipping the pressed bilayer's shapes into wall forming materials. An air suspension coating procedure which includes suspending and tumbling the two layers in current of air until the wall forming composition surrounds the layers is also used to form the semi-permeable wall of the osmotic dosage forms.

The dispenser of the osmotic pump dosage form may be in the form of a capsule. The capsule may comprise an osmotic hard capsule and/or an osmotic soft capsule. The osmotic hard capsule may be composed of two parts, a cap and a body, which are fitted together after the larger body is filled with the carboxylic acid losartan. The osmotic hard capsule may be fitted together by slipping or telescoping the cap section over the body section, thus completely surrounding and encapsulating the rosiglitazone. Hard capsules may be made by techniques known in the art.

The soft capsule of the osmotic pump dosage form may be a one-piece osmotic soft capsule. Generally, the osmotic soft capsule is of sealed construction encapsulating the carboxylic acid losartan. The soft capsule may be made by various processes, such as the plate process, the rotary die process, the reciprocating die process, and the continuous process.

Materials useful for forming the capsule of the osmotic pump dosage form are commercially available materials including gelatin, gelatin having a viscosity of about 5 to about 30 millipoises and a bloom strength up to about 150 grams; gelatin having a bloom value of about 160 to about 250; a composition comprising gelatin, glycerine, water and titanium dioxide; a composition comprising gelatin, erythrosin, iron oxide and titanium dioxide; a composition comprising gelatin, glycerine, sorbitol, potassium sorbate and titanium dioxide; a composition comprising gelatin, acacia, glycerin, and water; and the like, and combinations comprising one or more of the foregoing materials.

The semipermeable wall forming composition can be applied to the exterior surface of the capsule in laminar arrangement by molding, forming, air spraying, dipping or brushing with a semipermeable wall forming composition. Other techniques that can be used for applying the semipermeable wall are the air suspension procedure and the pan coating procedures. The air suspension procedure includes suspending and tumbling the capsule arrangement in a current of air and a semipermeable wall forming composition until the wall surrounds and coats the capsule. The procedure can be repeated with a different semipermeable wall forming composition to form a semipermeable laminated wall.

Exemplary solvents suitable for manufacturing the semipermeable wall include inert inorganic and organic solvents that do not adversely harm the materials, the capsule wall, the carboxylic acid losartan, the thermo-responsive composition, the expandable member, or the final dispenser. Solvents for manufacturing the semipermeable wall may be aqueous solvents, alcohols, ketones, esters, ethers, aliphatic hydrocarbons, halogenated solvents, cycloaliphatics, aromatics, heterocyclic solvents, and combinations comprising one or more of the foregoing solvents. Particular solvents include acetone, diacetone alcohol, methanol, ethanol, isopropyl alcohol, butyl alcohol, methyl acetate, ethyl acetate, isopropyl acetate, n-butyl acetate, methyl isobutyl ketone, methyl propyl ketone, n-hexane, n-heptane, ethylene glycol monoethyl ether, ethylene glycol monoethyl acetate, methylene dichloride, ethylene dichloride, propylene dichloride, carbon tetrachloride, nitroethane, nitropropane, tetrachloroethane, ethyl ether, isopropyl ether, cyclohexane, cyclooctane, benzene, toluene, naphtha, 1,4-dioxane, tetrahydrofuran, water, and mixtures thereof such as acetone and water, acetone and methanol, acetone and ethyl alcohol, methylene dichloride and methanol, and ethylene dichloride, methanol, and combinations comprising one or more of the foregoing solvents. The semipermeable wall may be applied at a temperature a few degrees less than the melting point of the thermo-responsive composition. Alternatively, the thermo-responsive composition can be loaded into the dispenser after applying the semipermeable wall.

The exit means or hole in the osmotic pump dosage form, for releasing the carboxylic acid losartan, can be formed by mechanical or laser drilling, or by eroding an erodible element in the wall, such as a gelatin plug. The orifice can be a polymer inserted into the semipermeable wall, which polymer is a porous polymer and has at least one pore, or which polymer is a microporous polymer and has at least one micro-pore.

In another embodiment, a carboxylic acid losartan dosage form comprises a floating or buoyant dosage form. The principle of a floating system is that the density of floating system is lower than that of gastric fluid. Floating of the dosage form allows for extended gastric residence time of the active agent and subsequent increases in bioavailability. Floating dosage forms are hydrodynamically balanced to have a bulk density (specific gravity) of less than one in contact with gastric fluid and which, therefore, will remain floating in gastric fluid. In some embodiments, a floating dosage form can also have controlled-release properties.

In one embodiment, a floating dosage form is a sustained-release formulation comprising a homogeneous mixture of carboxylic acid losartan with one or more hydrophillic hydrocolloids which, in contact with gastric fluid at body temperature, will form a soft gelatinous mass on the surface of the tablet, thus causing it to enlarge somewhat and acquire a bulk density (specific gravity) of less than one. Hydrocolloids suitable for use in the sustained-release formulations include one or more natural, partially or totally synthetic anionic or, preferably, nonionic hydrophillic gums, modified cellulosic substances or proteinaceous substances such as, for example, acacia, gum tragacanth, locust bean gum, guar gum, karaya gum, agar, pectin, carrageen, soluble and insoluble alginates, methylcellulose, hydroxypropylmethylcellulose, hydroxypropylcellulose, hydroxyethylcellulose, sodiumcarboxymethylcellulose, carboxypolymethylene, gelatin, casein, zein, bentonie, and the like. A preferred hydrocolloid is hydroxypropylmethylcellulose.

Edible, pharmaceutically inert, fatty materials having a specific gravity of less than one can be added to the floating formulation to decrease the hydrophillic property of the formulation and also to increase buoyancy. Examples of such materials include a purified grade of beeswax; fatty acids; long chain fatty alcohols such as, for example, cetyl alcohol, myristyl alcohol, stearyl alcohol, glycerides such as glyceryl esters of fatty acids or hydrogenated aliphatic acids such as, for example, glyceryl monostearate, glyceryl distearate, glyceryl esters of hydrogenated castor oil and the like; oils such as mineral oil and the like. The floating dosage forms may also include excipients, preservatives, stabilizers, tabletting lubricants and the like.

In one embodiment, the carboxylic acid losartan dosage form is a liquisolid dosage form. The term “liquisolid” refers to powdered forms of liquid medications formulated by converting solutions of water-insoluble solid active agents in suitable non- volatile solvent systems, into “dry” (i.e., dry-looking), nonadherent, free-flowing and readily compressible liquid/powder admixtures by blending with selected carrier and coating materials. Liquisolid systems comprising insoluble active agents may be classified into two subgroups: “powdered active agent solutions” and “powdered active agent suspensions”. These systems may be produced from the conversion of active agent solutions or suspensions into liquisolid systems. When non-volatile solvents are used to prepare the active agent solution or suspension, the liquid vehicle does not evaporate and thus, the active agent is carried within the liquid system which in turn, is dispersed throughout the final product.

In one embodiment, the carboxylic acid losartan is in particulate form. The carboxylic acid losartan in particulate form comprises nanoparticulate, micronized carboxylic acid losartan, or larger particles.

In one embodiment, the carboxylic acid losartan is in micronized form. The expression “in micronized form” means a substance having “an effective average particle size of less than about 20 μm”, meaning that at least 50% of the active agent particles, (e.g., carboxylic acid losartan particles) have a particle size of less than the average, by weight. Advantageously, the effective average particle size is less than 10 μm.

The carboxylic acid losartan is optionally micronized in the presence of a surfactant. Suitable surfactants include, for example, amphoteric, non-ionic, cationic or anionic surfactants. Examples of such surfactants are: sodium lauryl sulfate, monooleate, monolaurate, monopalmitate, monostearate or another ester of polyoxyethylene sorbitane, sodium dioctylsulfosuccinate (DOSS), lecithin, stearylic alcohol, cetostearylic alcohol, cholesterol, polyoxyethylene ricin oil, polyoxyethylene fatty acid glycerides, poloxamer®, and combinations comprising one or more of the foregoing surfactants.

The micronized carboxylic acid losartan optionally further comprises a hydrophilic polymer. “Hydrophilic polymer” means a high molecular weight substance (greater, for example, than 300 Da) having sufficient affinity towards water to dissolve therein and form a gel. Examples of such polymers are polyvinylpyrrolidone, poly(vinyl alcohol), hydroxypropylcellulose, hydroxymethylcellulose, hydroxypropylmethylcellulose, gelatin, and combinations comprising one or more of the foregoing polymers. The carboxylic acid losartan can be micronized in the presence of a hydrophilic polymer, or optionally micronized and then mixed with a hydrophilic polymer.

The micronized carboxylic acid losartan is optionally disposed on an inert hydrosoluble carrier. “Inert hydrosoluble carrier” means an excipient, generally hydrophilic, pharmaceutically inert, crystalline or amorphous, in a particulate form, not leading to a chemical reaction under the operating conditions employed, and which is soluble in an aqueous medium, notably in a gastric acid medium. Examples of such excipients are derivatives of sugars, such as lactose, saccharose, hydrolyzed starch (malto-dextrin), and combinations comprising one or more of the foregoing pharmaceutically acceptable carriers. Mixtures are also suitable. The individual particle size of the inert hydrosoluble carrier can be, for example, between 50 and 500 microns.

In one embodiment, a micronized carboxylic acid losartan composition is formed by spraying a suspension of carboxylic acid losartan micronized with a hydrophilic polymer onto an inert carrier. Following granulation, the granulate formed comprises crystals of, for example, lactose, which are isolated (or possibly agglomerated together by the spray solution) and particles of active ingredient and PVP adhering to the crystal surface. The granule could similarly be constituted of coated crystals which are agglomerated, or even of such an agglomerate having received a coating.

The micronized carboxylic acid losartan compositions can also be prepared by other methods, for example, by spraying a solution of the micronized active ingredient onto the hydrosoluble inert carrier.

The granulates thus obtained can, if desired, be provided with an outer coating or compressed into tablets, or form agglomerates.

In one embodiment, a nanoparticulate carboxylic acid losartan composition has an average particle size of less than about 2000 nm (i.e., 2 microns), less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, or less than 400 nm, as measured by light-scattering methods, microscopy, or other appropriate methods. By “an effective average particle size of less than about 2000 nm” it is meant that at least 50% of the active agent particles, (e.g., carboxylic acid losartan particles) have a particle size of less than the average, by weight, i.e., less than about 2000 nm, 1900 nm, 1800 nm, etc., when measured by the above-noted techniques. Preferably, at least about 70%, about 90%, or about 95% of the particles have a particle size of less than the effective average, i.e., less than about 2000 nm, 1900 nm, 1800 nm, 1700 nm, etc. As is understood in the art, the value for D50 of a nanoparticulate active agent is the particle size below which 50% of the particles fall, by weight. Similarly, D90 is the particle size below which 90% of the fibrate particles fall, by weight.

In one embodiment, a nanoparticulate carboxylic acid losartan dosage form comprises carboxylic acid losartan particles and at least one surface stabilizer. Useful surface stabilizers which can be employed include, but are not limited to, nonionic, anionic, cationic, ionic, and zwitterionic surfactants. Suitable surfactants include those listed below for use in amorphous formulations.

The concentration of the carboxylic acid losartan in the carboxylic acid losartan nanoparticles can be about 99.5% to about 0.001%, about 95% to about 0.1%, or about 90% to about 0.5%, by weight, based on the total combined weight of the carboxylic acid losartan and at least one surface stabilizer, not including other excipients. The concentration of the at least one surface stabilizer can be about 0.5% to about 99.999%, about 5.0% to about 99.9%, or about 10% to about 99.5%, by weight, based on the total combined dry weight of the carboxylic acid losartan and at least one surface stabilizer, not including other excipients.

The particulate carboxylic acid losartan compositions can be made using, for example, milling, homogenization, or precipitation techniques.

Milling carboxylic acid losartan to obtain a nanoparticulate dispersion comprises dispersing the carboxylic acid losartan particles in a liquid dispersion medium in which the carboxylic acid losartan is poorly soluble, followed by applying mechanical means in the presence of grinding media to reduce the particle size of the carboxylic acid losartan to the desired effective average particle size. The dispersion medium can be, for example, water, safflower oil, ethanol, t-butanol, glycerin, polyethylene glycol (PEG), hexane, glycol, or a combination comprising one or more of the foregoing media. In one embodiment, the dispersion medium is water.

The carboxylic acid losartan particles can be reduced in size in the presence of at least one surface stabilizer. Alternatively, the carboxylic acid losartan particles can be contacted with one or more surface stabilizers after attrition. Other compounds, such as a diluent, can be added to the carboxylic acid losartan/surface stabilizer composition during the size reduction process. Dispersions can be manufactured continuously or in a batch mode.

In one embodiment, a mixture of carboxylic acid losartan and one or more surface stabilizers is heated during the milling process. If a polymeric surface stabilizer is utilized, the temperature is raised to above the cloud point of the polymeric surface stabilizer but below the actual or depressed melting point of the carboxylic acid losartan. The utilization of heat may be important for scale up of the milling process, as it can aid in the solubilization of the one or more active agents.

Another method of forming the desired particulate carboxylic acid losartan composition is by microprecipitation. This is a method of preparing stable dispersions of poorly soluble active agents in the presence of one or more surface stabilizers and one or more colloid stability enhancing surface active agents free of trace toxic solvents or solubilized heavy metal impurities. Such a method comprises, for example: (1) dissolving carboxylic acid losartan in a suitable solvent; (2) adding the formulation from step (1) to a solution comprising at least one surface stabilizer; and (3) precipitating the formulation from step (2) using an appropriate non-solvent. The method can be followed by removal of any formed salt, if present, by dialysis or diafiltration and concentration of the dispersion by conventional means.

Homogenization methods include dispersing particles of carboxylic acid losartan, in a liquid dispersion medium, followed by subjecting the dispersion to homogenization to reduce the particle size of the carboxylic acid losartan to the desired effective average particle size. The carboxylic acid losartan can be reduced in size in the presence of at least one surface stabilizer. Alternatively, the carboxylic acid losartan particles can be contacted with one or more surface stabilizers either before or after attrition. Other compounds, such as a diluent, can be added to the carboxylic acid losartan/surface stabilizer composition either before, during, or after the size reduction process. Dispersions can be manufactured continuously or in a batch mode.

In another embodiment, the dosage form comprises amorphous carboxylic acid losartan. Amorphous solids are disordered arrangements of molecules that do not possess a distinguishable crystal lattice. In one embodiment, amorphous carboxylic acid losartan is formed by dissolving carboxylic acid losartan in a solvent in the presence of a polymer and optionally a surfactant, and evaporating the solvent to produce amorphous. In one embodiment, amorphous carboxylic acid losartan comprises carboxylic acid losartan, a polymer and a surfactant.

In another embodiment, a spray-drying process is used to form amorphous carboxylic acid losartan. In this embodiment, the carboxylic acid losartan, polymer and optional surfactant are dissolved in a solvent and then sprayed in a spray-drying apparatus where the solvent is rapidly evaporated, forming solid particles of amorphous carboxylic acid losartan. The term “spray-drying” broadly refers to processes involving breaking up liquid mixtures into small droplets (atomization) and rapidly removing solvent from the mixture in a spray-drying apparatus where there is a strong driving force for evaporation of solvent from the droplets. The strong driving force for solvent evaporation is generally provided by maintaining the partial pressure of solvent in the spray-drying apparatus well below the vapor pressure of the solvent at the temperature of the drying droplets. This is accomplished by (1) maintaining the pressure in the spray-drying apparatus at a partial vacuum (e.g., 0.01 to 0.50 atmospheres (atm); or (2) mixing the liquid droplets with a warm drying gas; or (3) both (1) and (2). In addition, at least a portion of the heat required for evaporation of solvent may be provided by heating the spray solution.

Solvents suitable for spray-drying are those in which the active agent and polymer are mutually soluble. Suitable solvents include, for example, alcohols such as methanol, ethanol, n-propanol, iso-propanol, and butanol; ketones such as acetone, methyl ethyl ketone and methyl iso- butyl ketone; esters such as ethyl acetate and propylacetate; and various other solvents such as acetonitrile, methylene chloride, toluene, THF, cyclic ethers, and 1,1,1-trichloroethane. Lower volatility solvents such as dimethyl acetamide or dimethylsulfoxide can also be used.

Suitable pharmaceutically acceptable polymers include, for example, hydroxypropyl cellulose, methyl cellulose, hydroxypropylmethyl cellulose, carboxymethyl cellulose, sodium carboxymethyl cellulose, cellulose acetate phthalate, cellulose acetate butyrate, hydroxyethyl cellulose, ethyl cellulose, polyvinyl alcohol, polypropylene, dextrans, dextrins, hydroxypropyl-beta-cyclodextrin, chitosan, co(lactic/glycolid) copolymers, poly(orthoester), poly(anhydrate), polyvinyl chloride, polyvinyl acetate, ethylene vinyl acetate, lectins, carbopols, silicon elastomers, polyacrylic polymers, maltodextrins, polyvinylpyrrolidone (PVP), polyethylene glycol (PEG), and alpha-, beta-, and gamma-cyclodextrins, and combinations comprising one or more of the foregoing polymers.

Suitable nonionic surfactants include, for example, polyoxyethylene fatty alcohol ethers (Macrogol and Brij), polyoxyethylene sorbitan fatty acid esters (Polysorbates), polyoxyethylene fatty acid esters (Myrj), sorbitan esters (Span), glycerol monostearate, polyethylene glycols, polypropylene glycols, cetyl alcohol, cetostearyl alcohol, stearyl alcohol, aryl alkyl polyether alcohols, polyoxyethylene-polyoxypropylene copolymers (poloxamers), poloxamines, methylcellulose, hydroxymethylcellulose, hydroxypropylcellulose, hydroxypropylmethylcellulose, noncrystalline cellulose, polysaccharides including starch and starch derivatives such as hydroxyethylstarch (HES), polyvinyl alcohol, polyvinylpyrrolidone, and combinations comprising one or more of the foregoing surfactants. In one embodiment, the nonionic surfactant is a polyoxyethylene and polyoxypropylene copolymer such as a block copolymer of propylene glycol and ethylene glycol.

Suitable anionic surfactants include but are not limited to alkyl sulfonates, alkyl phosphates, alkyl phosphonates, potassium laurate, triethanolamine stearate, sodium lauryl sulfate, sodium dodecylsulfate, alkyl polyoxyethylene sulfates, sodium alginate, dioctyl sodium sulfosuccinate, phosphatidyl choline, phosphatidyl glycerol, phosphatidyl inosine, phosphatidylserine, phosphatidic acid and their salts, glyceryl esters, sodium carboxymethylcellulose, cholic acid and other bile acids (e.g., cholic acid, deoxycholic acid, glycocholic acid, taurocholic acid, glycodeoxycholic acid), salts thereof (e.g., sodium deoxycholate, etc.), and combinations comprising one or more of the foregoing surfactants.

Suitable cationic surfactants include but are not limited to quaternary ammonium compounds, such as benzalkonium chloride, cetyltrimethylammonium bromide, chitosans, lauryldimethylbenzylammonium chloride, acyl camitine hydrochlorides, alkyl pyridinium halides, and combinations comprising one or more of the foregoing surfactants.

The carboxylic acid losartan solution feed can be spray-dried under a wide variety of conditions to yield amorphous carboxylic acid losartan. For example, various types of nozzles can be used to atomize the spray solution, thereby introducing the spray solution into the spray-dry chamber as a collection of small droplets. A suitable type of nozzle may be used to spray the solution as long as the droplets that are formed are sufficiently small that they dry sufficiently (due to evaporation of solvent) and preferably do not stick to or coat the spray-drying chamber wall.

The solution can be delivered to the spray nozzle or nozzles at a wide range of temperatures and flow rates. Generally, the solution temperature is just above the solvent's freezing point to about 20° C. above its ambient pressure boiling point (by pressurizing the solution) and in some cases even higher. Solution flow rates to the spray nozzle can vary over a wide range depending on the type of nozzle, spray- dryer size and spray-dry conditions such as the inlet temperature and flow rate of the drying gas. Generally, the energy for evaporation of solvent from the solution in a spray-drying process comes primarily from the drying gas.

The drying gas is a suitable gas, but for safety reasons, an inert gas such as nitrogen, nitrogen-enriched air or argon is preferably utilized. The drying gas is typically introduced into the drying chamber at a temperature between about 60° C. and about 300° C. and preferably between about 80° C. and about 240° C.

The large surface-to-volume ratio of the droplets and the large driving force for evaporation of solvent leads to rapid solidification times for the droplets. Solidification times should be less than about 20 seconds, less than about 10 seconds, or less than 1 second.

Following formation, the amorphous carboxylic acid losartan can be dried to remove residual solvent using a suitable drying process, such as tray drying, fluid bed drying, microwave drying, belt drying, rotary drying, and other drying processes known in the art. The final residual solvent level may be, for example, less than 1 wt %, preferably less than 0.1 wt %.

Once the amorphous carboxylic acid losartan has been formed, several processing operations can be used to facilitate incorporation of the amorphous carboxylic acid losartan into a dosage form. These processing operations include drying, granulation, and milling.

In another embodiment, a carboxylic acid losartan composition comprises a carboxylic acid losartan granulate comprising carboxylic acid losartan, a liquid surfactant and a solid, particulate filler.

A method of making a carboxylic acid losartan granulate comprises forming a carboxylic acid losartan solution by dissolving a quantity of carboxylic acid losartan in a surfactant heated to a temperature sufficient to melt the carboxylic acid losartan and form a carboxylic acid losartan solution; dispersing the carboxylic acid losartan solution onto solid, particulate filler, optionally in the presence of a binder to form a carboxylic acid losartan dispersion; cooling the carboxylic acid losartan dispersion at a temperature of less than about 15° C. to form a cooled carboxylic acid losartan dispersion; and granulating the cooled carboxylic acid losartan dispersion to form the carboxylic acid losartan granulate. As used herein, a liquid surfactant is a surfactant that is liquid at ambient temperatures and a solid surfactant is a surfactant that is a solid at ambient temperatures. The surfactant can be a liquid or a solid surfactant.

In one embodiment, the ratio of carboxylic acid losartan: surfactant is 1:1 to 10:1 on a per weight basis. A suitable liquid surfactant comprises, for example Tween 20, 40 and/or 80 (also called, polysorbate 80, or (polyoxyethylene 20 sorbitan monooleate)). Another example of the liquid surfactant is Triton X-100.

The surfactant is heated to a temperature sufficient to melt the carboxylic acid losartan and then the carboxylic acid losartan is added to the heated surfactant to produce a solution. The molten carboxylic acid losartan and surfactant are then dispersed onto a solid, particulate binder to form dispersed carboxylic acid losartan. Dispersion can be performed, for example, by mixing in a suitable apparatus such as a granulation apparatus, although granulation is not performed in this step. Exemplary particulate fillers include, for example, microcrystalline cellulose.

The carboxylic acid losartan can be dispersed on the filler optionally in the presence of a binder. Suitable binders include, for example, polyvinyl pyrrolidone, hydroxypropyl cellulose, hydroxypropyl methylcellulose, methylcellulose and hydroxyethyl cellulose, sugars, starch, and combinations comprising one or more of the foregoing binders.

After the dispersed carboxylic acid losartan is formed, the dispersed carboxylic acid losartan is cooled at a temperature of less than or equal to about 15° C immediately.

The cooled, dispersed carboxylic acid losartan is then granulated to produce the carboxylic acid losartan granulate. Suitable granulation techniques include, for example, wet granulation.

After granulation, the carboxylic acid losartan granulate is optionally mixed with a disintegrant, a lubricant, or other excipients, and compressed into tablets. Suitable disintegrants include, for example, low-substituted hydroxypropyl cellulose, cross-linked polyvinyl pyrrolidone (PVP-XL), sodium carboxymethylcellulose, e.g., Ac-di-sol®, sodium starch glycolate, sodium carboxymethyl starch, ion-exchange resins, starch, pregelatinized starch, and combinations comprising one or more of the foregoing disintegrants. Suitable lubricants include, for example, magnesium stearate.

In another embodiment, a carboxylic acid losartan dosage form comprises a liquid dosage form. Liquid pharmaceutically administrable compositions can, for example, be prepared by dissolving, dispersing, etc, fenofibic acid and optional pharmaceutical adjuvants in an excipient, such as, for example, water, saline, aqueous dextrose, glycerol, ethanol, olive oil, and other lipophilic solvents, and the like, to form a solution or suspension. If desired, the pharmaceutical composition to be administered may also contain minor amounts of nontoxic auxiliary substances, such as wetting or emulsifying agents, pH buffering agents, and the like, for example, sodium acetate, sorbitan monolaurate, triethanolamine sodium acetate, triethanolamine oleate, etc. Actual methods of preparing such dosage forms are known and will be apparent to those skilled in this art. The composition or formulation to be administered will contain an effective amount of an active compound of the invention.

In one embodiment, a carboxylic acid losartan dosage form comprises an emulsion or a microemulsion. Emulsions can be as a liquid administered directly into the patient's mouth from a measuring device, or within a soft, or a hard, gelatin capsule. Alternatively, emulsions can be adsorbed onto a carrier particle such as silicon dioxide and administered as a solid, oral dosage form, such as a tablet, granules, pellets or other multiparticulates, capsules that can contain the drug in the form of minitablets, beads, or a powder.

Emulsions and microemulsions comprise an oil phase, an aqueous phase, a surfactant and optionally a co-surfactant. Microemulsions differ from (macro or coarse) emulsions in that the dispersed phase consists of globules less than 100 nanometers (nm) (0.1 micrometers) and more particularly about 30 to about 60 nm in diameter. The differences between coarse emulsions and microemulsions, however, are not only one of size of the dispersed phase. Microemulsions do not separate on standing, whereas emulsions will separate, even though this may only occur after several years.

Active agent containing water-in-oil emulsions are, for example, made by dissolving a drug in a hydrophilic phase, and then mixing the solution with an oil, and eventually with an aqueous phase. Suitable oils include, for example, mono- , di- and triglycerides, fatty acids and their esters and esters of propylene glycol or other polyols. The fatty acids and esters used as such or where they form part of a glyceride may be short chain, medium chain or long chain. The ingredients may be of vegetable or animal origin, synthetic or semisynthetic. The oils include, but are not limited to natural oils, such as cottonseed oil, soybean oil, sunflower oil; canola oil; Captex® (various grades); Miglyol®; and Myvacet®.

Suitable surfactants, include, but are not limited to, various grades of the following commercial products: Arlacel®; Tween®; Capmul®; Centrophase®; Cremophor®; Labrafac®; Labrafil®; Labrasol®; Myverol®; and Tagat®. It is often unnecessary to include a co-surfactant in the microemulsion, when the microemulsion is formulated with the appropriate choice of low-HLB and high-HLB surfactants. However, where a co- surfactant is employed, the co-surfactant is preferably selected from non-toxic short and medium chain alcohols, but is not limited to these.

In another embodiment, a carboxylic acid losartan formulation comprises a formulation for transdermal administration. One can use topical administration to deliver carboxylic acid losartan by percutaneous passage of the drug into the systemic circulation of the patient. The skin sites include anatomic regions for transdermally administering the drug, such as the forearm, abdomen, chest, back, buttock, and mastoidal area. The carboxylic acid losartan is administered to the skin by placing on the skin either a topical formulation comprising the carboxylic acid losartan or a transdermal drug delivery device that administers the carboxylic acid losartan. In either embodiment, the delivery vehicle is designed, shaped, sized, and adapted for easy placement and comfortable retention on the skin.

A variety of transdermal drug delivery devices can be employed. For example, a simple adhesive patch comprising a backing material and an acrylate adhesive can be prepared. The carboxylic acid losartan and any penetration enhancer can be formulated into the adhesive casting solution. The adhesive casting solution can be cast directly onto the backing material or can be applied to the skin to form an adherent coating. See, e.g., U.S. Pat. Nos. 4,310,509; 4,560,555; and 4,542,012, incorporated herein by reference.

In other embodiments, the carboxylic acid losartan is delivered using a liquid reservoir system drug delivery device. These systems typically comprise a backing material, a membrane, an acrylate based adhesive, and a release liner. The membrane is sealed to the backing to form a reservoir. The drug or compound and any vehicles, enhancers, stabilizers, gelling agents, and the like are then incorporated into the reservoir. See, e.g., U.S. Pat. Nos. 4,597,961; 4,485,097; 4,608,249; 4,505,891; 3,843,480; 3,948,254; 3,948,262; 3,053,255; and 3,993,073; incorporated herein by reference.

Matrix patches comprising a backing, a drug/penetration enhancer matrix, a membrane, and an adhesive can also be employed to deliver carboxylic acid losartan transdermally. The matrix material typically comprises a polyurethane foam. The active agent, any enhancers, vehicles, stabilizers, and the like are combined with the foam precursors. The foam is allowed to cure to produce a tacky, elastomeric matrix which can be directly affixed to the backing material.

Also included are preparations for topical application to the skin comprising carboxylic acid losartan, typically in concentrations of about 0.001% to 10%, together with a non-toxic, pharmaceutically acceptable topical carrier. These topical preparations can be prepared by combining an active agent with conventional pharmaceutical diluents and pharmaceutically acceptable carriers commonly used in topical dry, liquid, and cream formulations. Ointments and creams may, for example, be formulated with an aqueous or oily base with the addition of suitable thickening and/or gelling agents. Such bases include water and/or an oil, such as liquid paraffin or a vegetable oil, such as peanut oil or castor oil. Thickening agents include soft paraffin, aluminum stearate, cetostearyl alcohol, propylene glycol, polyethylene glycols, woolfat, hydrogenated lanolin, beeswax, and the like.

Lotions may be formulated with an aqueous or oily base and will, in general, also include one or more of the following: stabilizing agents, emulsifying agents, dispersing agents, suspending agents, thickening agents, coloring agents, perfumes, and the like. Powders may be formed with the aid of a suitable powder base, e.g., talc, lactose, starch, and the like. Drops may be formulated with an aqueous base or non-aqueous base also comprising one or more dispersing agents, suspending agents, solubilizing agents, and the like.

The topical pharmaceutical compositions may also include one or more preservatives or bacteriostatic agents, e.g., methyl hydroxybenzoate, propyl hydroxybenzoate, chlorocreosol, benzalkonium chlorides, and the like.

### EXAMPLES

The following examples are given to illustrate the present invention. It should be understood, however, that the invention is not to be limited to the specific conditions or details described in these examples.

### Example 1

### Formulation for Composition with 50 mg CAL

The purpose for this example was to prepare a formulation for a composition comprising 50 mg CAL. Carboxylic acid losartan, magnesium carbonate, Avicel and povidone were placed in a 10 liter high shear gral mixer and mixed. While mixing, alcohol was added to form a first mixture. The first mixture was then discharged and dried in an oven under 50° C. until LOD (loss on drying) exhibited less than 2% using the conventional test method to form a dried first mixture. The dried first mixture was then milled and then transferred into a mixer. Povidone and magnesium stearate were added into the mixer and mixed with the dried first mixture to form a blend. The blend can be further processed into tablets using conventional compression technology or encapsulated into capsules, or any suitable dosage form. Details regarding this composition are presented in Table 2:

TABLE 2

| | Amount | |
|---|---|---|
| Ingredient | (mg/dosage) | % |
| Carboxylic Acid Losartan | 50 | 11.90 |
| Magnesium carbonate | 150 | 35.71 |
| Avicel PH 102 | 150 | 35.71 |
| Povidone S630 | 35 | 8.34 |
| Povidone XL 10 | 30 | 7.14 |
| Magnesium Stearate | 5 | 1.20 |
| Total | 420 | 100 |

### Example 2

### Formulation for Composition with 75 mg Carboxylic Acid Losartan

The purpose for this example was to prepare a formulation for a composition comprising 75 mg CAL. Carboxylic acid losartan, Avicel and the Povidones were screened through a 20 mesh screen into a 2 cu. ft. Gemco Blender and blended for 5 minutes without an intensifier bar. Magnesium stearate was screened through a 25 mesh screen into the same blender and mixed for 1 to 2 minutes to form a blend. The blend was discharged. The blend was further processed into tablets using conventional compression technology or encapsulated into capsules, or any suitable dosage form. Details regarding this composition are presented in Table 3:

TABLE 3

| Ingredient | Amount (mg/dosage) | % |
|---|---|---|
| Carboxylic acid losartan | 75 | 7.65 |
| Avicel PH 102 | 812 | 82.86 |
| Povidone S630 | 50 | 5.10 |
| Povidone XL 10 | 34 | 3.47 |
| Magnesium Stearate | 9 | 0.92 |
| Total | 980 | 100 |

### Example 3

### Formulation for Composition with 15 mg Carboxylic Acid Losartan

The purpose for this example was to prepare a formulation for a composition comprising 15 mg CAL. The composition was formed by mixing, granulating or blending the ingredients using a conventional wet or dry granulation process. The mixture can be further processed into tablets using conventional compression technology or encapsulated into capsules, or any suitable dosage form. Details regarding this composition are presented in Table 4:

TABLE 4

| | Amount | |
|---|---|---|
| Ingredient | (mg/dosage) | % |
| Carboxylic acid losartan | 15 | 5.08 |
| Sprayed mixture of Mannitol, | 165 | 55.93 |
| Sorbitol, Crospovidone and Silicon dioxide | | |
| Mannitol, USP | 100 | 33.90 |
| Entrapped Peppermint Flavor | 5 | 1.69 |
| Stearic Acid, NF | 10 | 3.40 |
| Total | 295 | 100 |

### Example 4

### Formulation for Composition with 10 mg Carboxylic Acid Losartan

The purpose for this example was to prepare a formulation for a composition comprising 10 mg CAL. The composition was formed by mixing, granulating or blending the ingredients using conventional wet or dry granulation process. The mixture can be further processed into tablets using conventional compression technology or encapsulated into capsules, or any suitable dosage form. Details regarding this composition are presented in Table 5:

TABLE 5

| Ingredient | Amount (mg/dosage) | % |
|---|---|---|
| Carboxylic acid losartan | 10 | 2.86 |
| HPMC K4MCR | 280 | 80.00 |
| Avicel pH 101 | 54 | 15.43 |
| Colloidal silicon dioxide | 2 | 0.57 |
| Magnesium stearate | 4 | 1.14 |
| Total | 350 | 100 |

### Example 5

### Formulation for Composition with 75 mg Carboxylic Acid Losartan

The purpose for this example was to prepare a delayed release formulation comprising a composition with 75 mg CAL. Carboxylic acid losartan was made into a core composition, as described in Example 2. The core was then coated with a delayed release functional coat. Optionally, the coated core may be coated with an additional cosmetic or functional coating. Details regarding the coating are presented in Table 6:

TABLE 6

| Ingredient | Amount (mg/dosage) |
|---|---|
| Eudragit L30D55 | 12-21 |
| TEC | 4-9 |
| Talc | 8 |

All patent and non-patent publications cited in this disclosure are incorporated herein by reference in its entirety to the extent they are not inconsistent with the instant disclosure. Further, even though the invention herein has been described with reference to particular examples and embodiments, it is to be understood that these examples and embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

What is claimed is:

1. A dosage form comprising a composition comprising a therapeutically effective amount of carboxylic acid losartan, its pharmaceutical salts, isomers, polymorphs, hydrates, solvates or metabolites.

2. The dosage form of claim 0 wherein the composition comprises a therapeutically effective amount of carboxylic acid losartan or its pharmaceutical salts.

3. The dosage form of claim 0 comprising between about 1 and about 120 mg of carboxylic acid losartan.

4. The dosage form of claim 0 wherein the composition further comprises a therapeutically effective amount of one or more additional therapeutic agents for the treatment of a disease or condition selected from the group consisting of hypertension, congestive heart failure, diabetic nephropathy, and myocardial infarction.

5. The dosage form of claim 0 formulated as an immediate release dosage form, and wherein administration of the composition results in AUC between about 185 and about 7920 ng.h/mL, Tmax between about 0.5 and about 6 hours, and Cₘₐₓ between about 25 and about 1000 ng/mL.

6. The dosage form of claim 0 formulated as a modified release dosage form, and wherein administration of the composition results in AUC between about 185 and about 7920 ng.h/mL, Tmax is between about 3 and about 14 hours, and Cₘₐₓ is between about 25 and about 800 ng/mL.

7. The dosage form of claim 2 characterized by being a solid dosage form comprising 50 mg of CAL, magnesium carbonate, Avicel PH 102, Povidone S630, Povidone XL 10, and magnesium stearate.

8. The dosage form of claim 2 characterized by being a solid dosage form comprising 75 mg of CAL, Avicel PH 102, Povidone S630, Povidone XL 10, and magnesium stearate.

9. The dosage form of claim 2 characterized by being a solid dosage form comprising 15 mg of CAL, mixture of mannitol, sorbitol, crospovidone, and silicon dioxide, mannitol, entrapped peppermint flavor, and stearic acid.

10. The dosage form of claim 2 characterized by being a solid dosage form comprising 10 mg of CAL and is formulated with HPMC K4MCR, Avicel PH 101, colloidal silicon dioxide, and magnesium stearate.

11. The dosage form of claim 2 comprising a solid dosage form characterized by being:
a core comprising 75 mg of CAL, Avicel PH 102, Povidone S630, Povidone XL 10, and magnesium stearate
wherein the core is coated with a coating comprising Eudragit L30D55, TEC and Talc.

12. A method of treating a condition selected from the group consisting of hypertension, congestive heart failure, diabetic nephropathy, and myocardial infarction, the method comprising administering a therapeutically effective amount of a composition comprising carboxylic acid losartan, its pharmaceutical salts, isomers, polymorphs, hydrates, solvates or metabolites.

13. The method of claim 12 wherein the composition comprises a therapeutically effective amount of a carboxylic acid losartan or its pharmaceutical salts.

14. The method of claim 12 wherein the composition further comprises one or more additional therapeutic agents for the condition being treated.

15. The method of claim 13 wherein the composition further comprises a cholesterol-lowering drug, a lipid-lowering drug, or both.

16. The method of claim 12 wherein the composition is formulated as a immediate release dosage form, and wherein administering composition results in AUC of between about 185 and 7920 ng.h/mL, Tmax of between about 0.5 and about 6 hours, and Cₘₐₓ of between about 25 and about 1000 ng/m L.

17. The method of claim 12 wherein the composition is formulated as a modified release dosage form, and wherein administering composition results in AUC of between about 185 and about 7920 ng.h/mL, Tmax of between about 3 and about 14 hours, and Cₘₐₓ of between about 25 and about 800 ng/mL.

18. The method of claim 12 wherein the carboxylic acid losartan dose administered to a patient is personalized based on individual patient's weight.

19. The method of claim 18 wherein between about 1 and about 70 mg of carboxylic acid losartan per dosage unit is administered to patients weighing less than 170 lbs, between about 3 and about 100 mg of carboxylic acid losartan per dosage unit is administered to patients weighing between about 150 and about 300 lbs, or between about 7 and about 120 mg of carboxylic acid losartan per dosage unit is administered to patients weighing more than 270 lbs.

20. A dosage form comprising a composition comprising a therapeutically effective amount of carboxylic acid losartan and a therapeutically effective amount of a cholesterol-lowering drug, a lipid-lowering drug, or both.

21. The dosage form of claim 20 comprising between about 1 and 100 mg of carboxylic acid losartan and a lipid-lowering drug.

22. The dosage form of claim 20 comprising between about 1 and about 100 mg of carboxylic acid losartan and a cholesterol-lowering drug.

23. The dosage form of claim 20 comprising between about 1 and about 90 mg of carboxylic acid losartan and a cholesterol-lowering drug and lipid-lowering drug.
